(19) United States
(12) Patent Application Publication
(10) Pub. No.: US 2015/0004157 A1
(43) Pub. Date: Jan. 1, 2015

(54) OSTEOPONTIN ISOFORM A INHIBITORS AND METHODS OF USE

(71) Applicants: Harvey I. Pass, Bronxville, NY (US); Jessica S. Donington, Pound Ridge, NY (US); Chandra M. Goparaju, Cliffside Park, NJ (US)

(72) Inventors: Harvey I. Pass, Bronxville, NY (US); Jessica S. Donington, Pound Ridge, NY (US); Chandra M. Goparaju, Cliffside Park, NJ (US)

(73) Assignee: NEW YORK UNIVERSITY, New York, NY (US)

(21) Appl. No.: 14/378,381

(22) PCT Filed: Feb. 13, 2013
(86) PCT No.: PCT/US2013/025816

§ 371 Date: Aug. 13, 2014

Related U.S. Application Data
(60) Provisional application No. 61/598,115, filed on Feb. 13, 2012.

(51) Int. Cl. A61K 38/19; C07K 14/52; A61K 45/06; C07K 16/24; C12N 15/115

(52) U.S. Cl. CPC A61K 38/19; C07K 16/24; C12N 15/115; A61K 45/06; C07K 14/52; C12N 2320/30; C07K 2317/76; C12N 2310/16
USPC 424/133.1; 530/326; 530/324; 530/387.9; 530/330; 536/23.1; 514/7.6; 424/649; 424/142.1; 424/94.6; 424/623; 424/139.1; 514/44 R

(57) ABSTRACT

The present invention relates to a therapeutic comprising an osteopontin isoform a (“OPNa”) inhibitor where the OPNa inhibitor blocks activity of extracellular OPNa exon 4. The OPNa inhibitor is selected from the group consisting of (i) an exon-4 specific antibody or binding portion thereof; (ii) a peptide mimic of OPNa exon 4 or a fragment thereof; (iii) a nucleic acid aptamer that specifically binds to OPNa exon 4 or a fragment thereof; and (iv) a peptide inhibitor that binds to OPNa exon 4 or a fragment thereof. The present invention also relates to methods of inhibiting tumor growth and/or metastasis in a subject, treating a subject with chemotherapeutic resistance, and methods of increasing tumor cell sensitivity to a cancer therapeutic by administering an OPNa inhibitor according to the present invention.

This application is a continuation of U.S. Provisional Patent Application Ser. No. 61/598,115, filed Feb. 13, 2012, which is hereby incorporated by reference in its entirety.

### FIELD OF THE INVENTION

The present invention relates generally inhibitors of osteopontin isoform a (“OPNa”), as well as therapeutics and methods of treatment including such inhibitors.

### BACKGROUND OF THE INVENTION

Lung cancer is the world's leading cause of cancer related death, and Non-small cell lung cancer (“NSCLC”) accounts for more than 80% of all lung cancer. NSCLC is associated with high rates of early metastasis, disease recurrence, and resistance to chemotherapies. NSCLC is an aggressive malignancy with early metastatic spread and high rates of recurrence. NSCLC also has a high rate of resistance to chemotherapies. For example, Cisplatin is the most efficacious and commonly used chemotherapy for NSCLC, but resistance is common. Osteopontin (“OPN”) is a secreted phosphoprotein involved in all stages of cancer progression including invasion, angiogenesis, and metastasis. OPN is a ubiquitous extracellular protein associated with a wide range of normal and pathologic functions, regulating cell matrix interactions and cellular signaling through binding of cell surface receptors. Extensively implicated as a regulator of metastatic function, OPN mediates cell adhesion, chemotaxis, angiogenesis, anchorage independent growth and avoidance of apoptosis in several solid tumors.

OPN is over-expressed in human NSCLC tumors compared to normal lung tissue (Hu et al., “Overexpression of Osteopontin is Associated With More Aggressive Phenotypes in Human Non-small Cell Lung Cancer,” Clin Cancer Res 11:4646-52 (2005)). Elevated OPN expression in NSCLC tumors is associated with increased stage, lymph node involvement and poor prognosis (Le et al., “An Evaluation of Tumor Oxygenation and Gene Expression in Patients With Early Stage Non-small Cell Lung Cancers,” Clin Cancer Res 12:1507-14 (2006); Mack et al., “Lower Osteopontin Plasma Levels are Associated With Superior Outcomes in Advanced Non-small-cell Lung Cancer Patients Receiving Platinum-based Chemotherapy: SWOG Study S0003,” J Clin Oncol 26:4771-6 (2008)). Its role as a regulator of metastatic function has been observed in numerous solid tumors, including NSCLC, but its underlying molecular mechanisms have not been fully characterized. Among these critical roles, OPN is a component of the mechanisms cancer cells use to elude apoptosis (Graessmann et al., “Chemotherapy Resistance of Mouse WAP-SVT/t Breast Cancer Cells is Mediated by Osteopontin, Inhibiting Apoptosis Downstream of Caspase-3,” Oncogene 26:2840-50 (2007); Courter et al., “The RGD Domain of Human Osteopontin Promotes Tumor Growth and Metastasis Through Activation of Survival Pathways,” PLoS One 5:e9633 (2010); Graves et al., “Hypoxia in Models of Lung Cancer: Implications for Targeted Therapeutics,” Clin Cancer Res 16:4843-52 (2010); Gu et al., “Osteopontin is Involved in the Development of Acquired Chemo-resistance of Cisplatin in Small Cell Lung Cancer,” Lung Cancer 66:176-83 (2009)). When cells are stressed by hypoxia, starvation or chemotherapy, OPN activates alternative pathways for survival (Courter et al., “The RGD Domain of Human Osteopontin Promotes Tumor Growth and Metastasis Through Activation of Survival Pathways,” PLoS One 5:e9633 (2010)).

The most widely studied OPN-cell surface binding interaction in NSCLC is that between OPN's central RGD domain. α_{ν}β₃-RGD binding is implicated in complex networks involved in NSCLC progression, metastasis formation, and increased survival through the PI3K/AKT pathways (Courter et al., “The RGD Domain of Human Osteopontin Promotes Tumor Growth and Metastasis Through Activation of Survival Pathways,” PLoS One 5:e9633 (2010); Fong et al., “Osteopontin Increases Lung Cancer Cells Migration via Activation of the Alphavbeta3 Integrin/FAK/Akt and NF-kappaB-dependent Pathway,” Lung Cancer 64:263-70 (2009); and Mi et al., “RNA Aptamer Blockade of Osteopontin Inhibits Growth and Metastasis of MDA-MB231 Breast Cancer Cells,” Mol Ther 17:153-61 (2009)). Other important OPN binding motifs include the SVVYGLR sequence adjacent to the RGD and the CD44 binding domain in the carboxyl region of the molecule. However, in each case, there is strong evidence for cross-talk and pathway regulation from additional OPN binding domains (Graessmann et al., “Chemotherapy Resistance of Mouse WAP-SVT/t Breast Cancer Cells is Mediated by Osteopontin, Inhibiting Apoptosis Downstream of Caspase-3,” Oncogene 26:2840-50 (2007); Courter et al., “The RGD Domain of Human Osteopontin Promotes Tumor Growth and Metastasis Through Activation of Survival Pathways,” PLoS One 5:e9633 (2010); and Gu et al., “Osteopontin is Involved in the Development of Acquired Chemo-resistance of Cisplatin in Small Cell Lung Cancer,” Lung Cancer 66:176-83 (2009)).

Despite these advances, there remains a continuing and pressing need for greater knowledge of the mechanisms underlying OPN's association with cancer progression and cell survival. Such discoveries would have great therapeutic potential in oncology, inflammation, vascular biology, and sepsis.

The present invention overcomes these and other deficiencies in the art.

### SUMMARY OF THE INVENTION

One aspect of the present invention relates to a therapeutic comprising an osteopontin isoform a (“OPNa”) inhibitor where the OPNa inhibitor blocks activity of extracellular OPNa exon 4. The OPNa inhibitor is selected from the group consisting of (i) an exon-4 specific antibody or binding portion thereof; (ii) a peptide mimic of OPNa exon 4 or a fragment thereof; (iii) a nucleic acid aptamer that specifically binds to OPNa exon 4 or a fragment thereof and (iv) a peptide inhibitor that binds to OPNa exon 4 or a fragment thereof.

Another aspect of the present invention relates to a method of inhibiting tumor growth and/or metastasis in a subject. This method includes selecting a subject having a tumor, where the tumor cells express OPNa exon 4 and administering to the selected subject an OPNa inhibitor.

Another aspect of the present invention relates to a method of inhibiting tumor growth and/or metastasis in a subject. This method includes selecting a subject having a tumor, where the tumor cells express OPNa exon 4 and administering to the selected subject an OPNa inhibitor and a cancer therapeutic.

Another aspect of the present invention relates to a method of treating a subject with chemotherapeutic resistance. This method includes selecting a subject having chemotherapeutic resistance and administering to the selected subject a OPNa inhibitor and a cancer therapeutic.

Yet a further aspect of the present invention relates to a method for increasing tumor cell sensitivity to a cancer therapeutic in a subject. The method includes selecting a subject in need of increased tumor cell sensitivity to a cancer therapeutic; providing an OPNa inhibitor; providing the cancer therapeutic; and administering to the subject the OPNa inhibitor and the cancer therapeutic under conditions effective to increase tumor cell sensitivity to the cancer therapeutic in the selected subject as compared to administration of the cancer therapeutic alone.

Surprisingly, disclosed here is a unique and undiscovered binding domain of OPN, which is essential to its important role in NSCLC progression. As described infra, a previously uncharacterized binding domain in exon 4 of OPN is identified that is essential for its malignant properties in NSCLC. Moreover, the Examples demonstrate that a peptide which mimics the central sequence of exon 4 engages the cell surface in an OPN-specific manner, blocks OPN cell surface binding, and abrogates malignant properties in NSCLC cells. The inventors have shown that exon 4 of OPN has a membrane-associated protein complex on NSCLC cells that is essential for the increased malignant properties conferred by OPNa. Thus, antagonizing this membrane complex and associated pathways will decrease NSCLC growth and increase chemotherapy sensitivity. The inventors show, inter alia, that inhibition of binding and regulatory activity of OPN exon 4 in NSCLC has significant therapeutic benefit. The discovery of the present invention has tremendous translational implications in oncology as well as in other systems where OPN has a central role, including wound healing, inflammation, vascular biology, and bone metabolism.

### BRIEF DESCRIPTION OF THE DRAWINGS

FIGS. 1A and 1B are schematic drawings that illustrate structural elements of OPN protein. FIG. 1A illustrates structural elements of OPN isoform a (e.g., SEQ ID NO:2), including central RGD and SVVYGLR regions and exon 4 in the amino terminus. FIG. 1B shows a schematic comparison of OPN isoforms a and c.

FIG. 2 shows OPN mRNA expression from NSCLC tumors (T) and paired normal lung (N) using primers that amplify each OPN isoform. OPN is over-expressed in 91% of tumors (all but specimen 2 here) and OPNa is the dominant isoform expressed in all tumors.

FIG. 3 shows OPN mRNA expression in NSCLC and immortalized bronchial epithelial cell lines from semiquantitative RT-PCR analysis using primers which amplify each isoform. High endogenous OPNa expression is seen in H460 and A549. Decreased expression is noted in H358 and none is seen in immortalized bronchial epithelial lines. Secreted OPN correlate with expression of the OPNa isoform (r=0.9120, P=0.0006) (adeno, adenocarcinoma; BrEpC, bronchial epithelial cell).

FIGS. 4A-4E are graphs of experimental results showing a 4-amino acid deletion (PDAV (SEQ ID NO:5) in exon 4 of OPN abolishes the malignant input of OPN in NSCLC cell lines (FIGS. 4A-4D) and increases cisplatin sensitivity (FIG. 4E). FIG. 4A is a bar graph that shows results of a proliferation assay. FIG. 4B is a bar graph that shows results of a migration assay. FIG. 4C is a bar graph that shows results of an anchorage independent growth assay. FIG. 4D is a bar graph that shows results demonstrating invasion of cells is increased in all cell lines with full length OPN expression, but not by OPNc that lacks exon 4 or by the PDAV-deleted mutant. FIG. 4E is a line graph of experimental results showing the PDAV-deleted mutant increases cisplatin sensitivity.

FIG. 5 is a schematic drawing illustrating the 17-amino acid peptide mimic (SEQ ID NO:4), and scrambled sham control WQKPAPLDTDPKVYANS (SEQ ID NO:23), of the central sequence of exon 4 (SEQ ID NO:3). The mimic includes the crucial PDAV (SEQ ID NO:5) sequence identified by site-directed mutagenesis.

FIGS. 6A-C show results of exon 4 peptide binding assays. FIG. 6A is a bar graph showing fluorescent labeled exon 4 peptide binds NSCLC cell surface, while scrambled sham does not (exon 4 peptide shown as two left bars and scrambled peptide shown as right two bars for each respective cell line). FIG. 6B is a line graph showing results that demonstrate exon 4 peptide is out-competed for binding at the cell surface with increasing doses of OPN (top line shows results for exon 4 peptide and bottom line shows results for the scrambled peptide). FIG. 6C is a bar graph showing results which demonstrate that recombinant OPNc (rOPN, Sunnylabs, Sittingbourne, UK) does not displace fluorescent labeled peptide from the NSCLC cell surface.

FIGS. 7A-7E are bar graphs (FIGS. 7A to 7D) and a line graph (FIG. 7E) showing results that demonstrate addition of exon 4 peptide to media inhibits invasion (FIGS. 7B and 7D) and proliferation (FIG. 7A) and increases apoptosis (FIG. 7C) in cells with high endogenous OPN expression (A549 and H460). (Bar graphs of FIGS. 7A-7C show results for sham as the far left bar, scrambled peptide as the middle bar, and exon 4 peptide as the right bar for each respective cell line.) The results also indicate that exon 4 peptide has less impact on same functions in H358 cells with low endogenous OPN expression. FIG. 7D additionally demonstrates both rOPNc and the exon 4 peptide decrease invasive capacity, and the addition of the exon 4 peptide to exogenous rOPNa blocks its impact on invasion. FIG. 7E shows that exon 4 peptide increased cisplatin sensitivity, as indicated by the 2-fold decrease in cell viability compared to untreated or scrambled peptide at cisplatin concentrations of 0.1-0.8 μM/ml.

FIGS. 8A-8B are bar graphs showing the results of a ligand sorting assay in which cells were FACS sorted into 3 groups based on density of fluorescent labeled peptide. The results are shown for cells used in invasion (FIG. 8A) and TUNEL (FIG. 8B) assays. The cells manifested increasing invasive properties and decreased apoptosis as a function of the density of exon 4 binding site. These results implicate increased malignant properties conferred through the putative complex.

FIGS. 9A-9D are bar graphs showing that exon 4 peptide abrogates OPN-mediated EMT in NSCLC cells lines. In particular, treatment with exon 4 peptide resulted in a significant decrease in the relative expression of TFGb1-r (FIG. 9A) and N-cadherin (FIG. 9B) and a significant increase in desmoplakin (FIG. 9C) and cytokeratin-20 (FIG. 9D) in cells with high endogenous OPN expression (A549 and H460). (Bar graphs of show results for sham as the far left bar, scrambled peptide as the middle bar, and exon 4 peptide as the right bar for each respective cell line.)

FIG. 10 is a schematic drawing illustrating a theoretical model indicating that malignant properties bestowed by OPN in NSCLC are dependent on exon 4 cell surface interactions and are decreased by steric hindrance by exon 4 peptide.

### DETAILED DESCRIPTION OF THE INVENTION

Implicated as a regulator of metastatic function, OPN mediates cell adhesion, chemotaxis, angiogenesis, anchorage independent growth and avoidance of apoptosis in several solid tumors. In NSCLC, OPN promotes an anti-apoptotic response in tumor cells under stress (Graessmann et al., “Chemotherapy Resistance of Mouse WAP-SVT/t Breast Cancer Cells is Mediated by Osteopontin, Inhibiting Apoptosis Downstream of Caspase-3,” Oncogene 26:2840-50 (2007); Courter et al., “The RGD Domain of Human Osteopontin Promotes Tumor Growth and Metastasis Through Activation of Survival Pathways,” PLoS One 5:e9633 (2010); Graves et al., “Hypoxia in Models of Lung Cancer: Implications for Targeted Therapeutics,” Clin Cancer Res 16:4843-52 (2010), which are hereby incorporated by reference in their entirety). OPNa, the full length protein, is disproportionately expressed in NSCLC tumors and cell lines, and expression is associated with increased aggressive behavior, cell survival, and epithelial-mesenchymal transition (EMT), (Saika et al. “Loss of Osteopontin Perturbs the Epithelial-mesenchymal Transition in an Injured Mouse Lens Epithelium,” Lab Invest 87:130-8 (2007) and Goparaju et al. “Functional Heterogeneity of Osteopontin Isoforms in Non-small Cell Lung Cancer,” J Thorac Oncol 5:1516-23 (2010), which are hereby incorporated by reference in their entirety) which has been strongly associated with cisplatin resistance in NSCLC (Zhuo et al., “Knockdown of Snail, a Novel Zinc Finger Transcription Factor, via RNA Interference Increases A549 Cell Sensitivity to Cisplatin via JNK/Mitochondrial Pathway,” Lung Cancer 62:8-14 (2008); Zhuo et al., “Short Interfering RNA Directed Against TWIST, a Novel Zinc Finger Transcription Factor, Increases A549 Cell Sensitivity to Cisplatin via MAPK/Mitochondrial Pathway,” Biochem Biophys Res Commun 369:1098-102 (2008); and Shintani et al., “Epithelial to Mesenchymal Transition is a Determinant of Sensitivity to Chemoradiotherapy in Non-small Cell Lung Cancer,” The Annals of Thoracic Surgery 92:1794-804 (2011) discussion 804, which are hereby incorporated by reference in their entirety). Conversely, the shortest variant, OPNc, which lacks exon 4, a 27 amino acid sequence in at the amino terminal, does not exhibit these protumorigenic properties. While it is established that interaction of the central RGD domain of OPN, plays a role in α_{ν}β₃ integrin engagement at the plasma membrane, little is known structurally or functionally about the 27 amino acid sequence of exon 4 which differentiates the two isoforms.

OPN is a component of the mechanisms cancer cells use to elude apoptosis (Graessmann et al., “Chemotherapy Resistance of Mouse WAP-SVT/t Breast Cancer Cells is Mediated by Osteopontin, Inhibiting Apoptosis Downstream of Caspase-3,” Oncogene 26:2840-50 (2007); Courter et al., “The RGD Domain of Human Osteopontin Promotes Tumor Growth and Metastasis Through Activation of Survival Pathways,” PLoS One 5:e9633 (2010); Graves et al., “Hypoxia in Models of Lung Cancer Implications for Targeted Therapeutics,” Clin Cancer Res 16:4843-52 (2010); Gu et al., “Osteopontin is Involved in the Development of Acquired Chemo-resistance of Cisplatin in Small Cell Lung Cancer,” Lung Cancer 66:176-83 (2009), which are hereby incorporated by reference in their entirety). When cells are stressed by hypoxia, starvation or chemotherapy, OPN activates alternative pathways for survival (Courter et al., “The RGD Domain of Human Osteopontin Promotes Tumor Growth and Metastasis Through Activation of Survival Pathways,” PLoS One 5:e9633 (2010), which is hereby incorporated by reference in its entirety). Therefore, OPN related pathways serve as promising targets for therapeutic inhibition, which may have potential to decrease cancer cell survival and improve the efficacy of commonly used chemotherapeutics including cisplatin.

As a secreted protein, OPN is an attractive candidate for therapeutic blockade due to its accessibility and because it is an important intermediate between tumors and their microenvironment (Anborgh et al., “Role of the Metastasis-promoting Protein Osteopontin in the Tumour Microenvironment,” J Cell Mol Med 14:2037-44 (2010) and Shevde et al., “Osteopontin: An Effector and an Effect of Tumor Metastasis,” Curr Mol Med 10:71-81 (2010), which are hereby incorporated by reference in their entirety). Specifically, the antagonism of OPN-receptor interactions may provide a readily therapeutic target to increase the sensitivity of tumor cells to commonly used chemotherapeutic regimes.

OPN belongs to the SIBLING family of proteins that regulate cell adhesion, migration, invasion, chemotaxis, and survival by binding to integrins and other cell surface receptors. OPN also plays an important role in a wide range of normal processes including wound healing, bone mineralization, and immune modulation. OPN is expressed in many tissues including bone, vascular tissue, kidney, epithelial cells, along with numerous tumors. The diverse physiologic and pathophysiologic roles for OPN are attributed to tissue specific post-translational modifications that generate unique forms and alter its function.

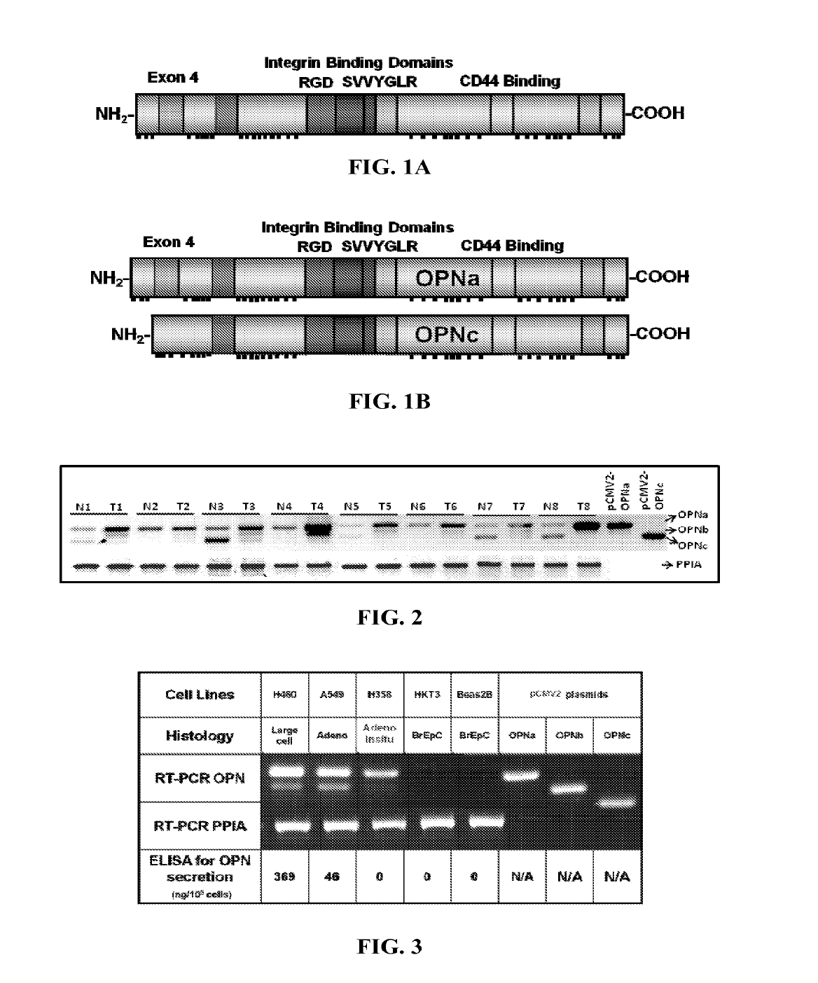

OPN binds to a number of cell surface receptors, including integrins and CD44 and indirectly influences the activity of growth factor receptors (Tuck et al., “Osteopontin Overexpression in Breast Cancer: Knowledge Gained and Possible Implications for Clinical Management,” Journal of Cellular Biochemistry 102:859-68 (2007) and Bellahcene et al., “Small Integrin-binding Ligand N-linked Glycoproteins (SIBLINGs): Multifunctional Proteins in Cancer,” Nat Rev Cancer 8:212-26 (2008), which are hereby incorporated by reference in their entirety). OPN lacks a complex secondary structure, but, as shown in FIGS. 1A and 1B, has a number of highly conserved structural elements including RGD (arginine-glycine-aspartate) and SVVYGLR (serine-valine-valine-glycine-leucine-arginine) domains for integrin binding, a calcium binding site, and heparin binding domains that mediate CD44 binding (Tuck et al., “Osteopontin Overexpression in Breast Cancer: Knowledge Gained and Possible Implications for Clinical Management,” Journal of Cellular Biochemistry 102:859-68 (2007), which is hereby incorporated by reference in its entirety).

Three distinct OPN splice variants are known in humans, but little was known about their unique functions until now (O'Regan et al., “Osteopontin: A Key Cytokine in Cell-mediated and Granulomatous Inflammation,” Int J Exp Pathol 81:373-90 (2000), which is hereby incorporated by reference in its entirety). OPNa encodes the full length protein, OPNb has a deletion of exon 5, and OPNc is defined by deletion of exon 4. All forms of OPN are subject to extensive post-translational modification in the form of phosphorylation, glycosylation, and tyrosine sulphation (Christensen et al., “Post-translationally Modified Residues of Native Human Osteopontin are Located in Clusters: Identification of 36 Phosphorylation and Five O-glycosylation Sites and Their Biological Implications,” The Biochemical Journal 390:285-92 (2005), which is hereby incorporated by reference in its entirety). These modifications are cell-type specific (Anborgh et al., “New Dual Monoclonal ELISA for Measuring Plasma Osteopontin as a Biomarker Associated With Survival in Prostate Cancer: Clinical Validation and Comparison of Multiple ELISAs,” Clinical Chemistry 55:895-903 (2009) and Zhang et al., “An Integrated Procedure of Selective Injection, Sample Stacking and Fractionation of Phosphopeptides for MALDI MS Analysis,” Anal Chim Acta 581:268-80 (2007), which are hereby incorporated by reference in their entirety), but their impact on OPN structure and likely impact function are not fully understood.

It has been demonstrated that the three naturally occurring OPN splice variants are differentially expressed in NSCLC, with the full length isoform, OPNa, being preferentially up-regulated in tumors compared to normal tissue (Goparaju et al. “Functional Heterogeneity of Osteopontin Isoforms in Non-small Cell Lung Cancer,” J Thorac Oncol 5:1516-23 (2010) and Blasberg et al., “Lung Cancer Osteopontin Isoforms Exhibit Angiogenic Functional Heterogeneity,” The Journal of Thoracic and Cardiovascular Surgery 139:1587-93 (2010), which are hereby incorporated by reference in their entirety). Similar patterns of divergent OPN isoform expression are seen in hepatocellular carcinoma (Chac et al., “Osteopontin Splice Variants Differentially Modulate the Migratory Activity of Hepatocellular Carcinoma Cell Lines,” Int J Oncol 35:1409-16 (2009), which is hereby incorporated by reference in its entirety), head and neck carcinomas (Courter et al., “The RGD Domain of Human Osteopontin Promotes Tumor Growth and Metastasis Through Activation of Survival Pathways,” PLoS One 5:e9633 (2010), which is hereby incorporated by reference in its entirety), and mesothelioma (Ivanov et al., “Tumorigenic Properties of Alternative Osteopontin Isoforms in Mesothelioma,” Biochem Biophys Res Commun 382:514-8 (2009), which is hereby incorporated by reference in its entirety), while the c-isoform is the over-expressed variant in breast (Mirza et al., “Osteopontin-c is a Selective Marker of Breast Cancer,” International Journal of Cancer 122:889-97 (2008) and Weber et al., “Osteopontin is a Marker for Cancer Aggressiveness and Patient Survival,” British Journal of Cancer 103:861-9 (2010), which is hereby incorporated by reference in its entirety), ovarian (Tilli et al., “Osteopontin-c Splicing Isoform Contributes to Ovarian Cancer Progression,” Mol Cancer Res 9:280-93 (2011), which is hereby incorporated by reference in its entirety), and prostate cancers (Tilli et al., “Expression Analysis of Osteopontin mRNA Splice Variants in Prostate Cancer and Benign Prostatic Hyperplasia,” Exp Mol Pathol 92:13-9 (2011), which is hereby incorporated by reference in its entirety). Plasma OPN levels are known to be elevated in NSCLC and increased levels are associated with poor prognosis in early and advanced stage NSCLC (Mack et al., “Lower Osteopontin Plasma Levels are Associated With Superior Outcomes in Advanced Non-small-cell Lung Cancer Patients Receiving Platinum-based Chemotherapy: SWOG Study S0003,” J Clin Oncol 26:4771-6 (2008) and Blasberg et al., “Reduction of Elevated Plasma Osteopontin Levels With Resection of Non-small-cell Lung Cancer,” J Clin Oncol 28:936-41 (2010), which are hereby incorporated by reference in their entirety). Elevated plasma OPN levels in NSCLC have recently been attributed to increases in the full length isoform alone (Wu et al., “Identification and Quantification of Osteopontin Splice Variants in the Plasma of Lung Cancer Patients Using Immunoaffinity Capture and Targeted Mass Spectrometry,” Biomarkers (2011), which is hereby incorporated by reference in its entirety), further implicating the a-isoform as the malignant culprit in NSCLC.

The present inventors have uncovered a unique and undiscovered binding domain of OPN which is important to its role in NSCLC progression. Strikingly, although exon 4 has been implicated in OPN-mediated lymphocyte activation in rheumatoid arthritis (Cao et al., “A Novel Functional Motif of Osteopontin for Human Lymphocyte Migration and Survival,” Mol Immunol 45:3683-92 (2008) and Dai et al., “A Functional Motif QLYxxYP is Essential for Osteopontin Induced T Lymphocyte Activation and Migration,” Biochem Biophys Res Commun 380:715-20 (2009), which are hereby incorporated by reference in their entirety), it has not been previously recognized as an important regulatory sequence for OPN in oncology. The malignant properties conferred by this relatively unexplored site of OPNa have not yet been reported.

One aspect of the present invention relates to a therapeutic comprising an osteopontin isoform a (“OPNa”) inhibitor where the OPNa inhibitor blocks activity of extracellular OPNa exon 4. The OPNa inhibitor is selected from the group consisting of (i) an exon-4 specific antibody or binding portion thereof; (ii) a peptide mimic of OPNa exon 4 or a fragment thereof; (iii) a nucleic acid aptamer that specifically binds to OPNa exon 4 or a fragment thereof; and (iv) a peptide inhibitor that binds to OPNa exon 4 or a fragment thereof.

Another aspect of the present invention relates to an OPNa inhibitor according to the present invention that is not in the form of a therapeutic.

In one embodiment of the present invention, the OPNa inhibitor blocks OPNa exon 4 cell surface receptor binding activity. In one embodiment, the cell surface receptor is on the surface of a tumor cell. In one particular embodiment, the cell surface receptor is on the surface of a NSCLC cell.

It will be understood that inhibition of extracellular OPNa exon 4 relates to inhibition of OPNa exon 4 that is present outside of the cell. This can include OPNa or a fragment thereof that is exposed to the extracellular face of the cell or OPNa that has been secreted to the extracellular space.

In one embodiment of the present invention OPNa is encoded by the nucleotide sequence of SEQ ID NO:1 (GenBank Accession No. NM_{—}001040058, which is hereby incorporated by reference in its entirety), as follows:

| | |
|---|---|
| 1 | ctccctgtgt tggtggagga tgtctgcagc agcatttaaa ttctgggagg gcttggttgt |
| 61 | cagcagcagc aggaggaggc agagcacagc atcgtcggga ccagactcgt ctcaggccag |
| 121 | ttgcagcctt ctcagccaaa cgccgaccaa ggaaaactca ctaccatgag aattgcagtg |
| 181 | atttgctttt gcctcctagg catcacctgt gccataccag ttaaacaggc tgattctgga |
| 241 | agttctgagg aaaagcagct ttacaacaaa tacccagatg ctgtggccac atggctaaac |
| 301 | cctgacccat ctcagaagca gaatctccta gccccacaga atgctgtgtc ctctgaagaa |
| 361 | accaatgact ttaaacaaga gacccttcca agtaagtcca acgaaagcca tgaccacatg |
| 421 | gatgatatgg atgatgaaga tgatgatgac catgtggaca gccaggactc cattgactcg |
| 481 | aacgactctg atgatgtaga tgacactgat gattctcacc agtctgatga gtctcaccat |
| 541 | tctgatgaat ctgatgaact ggtcactgat tttcccacgg acctgccagc aaccgaagtt |
| 601 | ttcactccag ttgtccccac agtagacaca tatgatggcc gaggtgatag tgtggtttat |
| 661 | ggactgaggt caaaatctaa gaagtttcgc agacctgaca tccagtaccc tgatgctaca |
| 721 | gacgaggaca tcacctcaca catggaaagc gaggagttga atggtgcata caaggccatc |
| 781 | cccgttgccc aggacctgaa cgcgccttct gattgggaca gccgtgggaa ggacagttat |
| 841 | gaaacgagtc agctggatga ccagagtgct gaaacccaca gccacaagca gtccagatta |
| 901 | tataagcgga aagccaatga tgagagcaat gagcattccg atgtgattga tagtcaggaa |
| 961 | ctttccaaag tcagccgtga attccacagc catgaatttc acagccatga agatatgctg |
| 1021 | gttgtagacc ccaaaagtaa ggaagaagat aaacacctga aatttcgtat ttctcatgaa |
| 1081 | ttagatagtg catcttctga ggtcaattaa aaggagaaaa aatacaattt ctcactttgc |
| 1141 | atttagtcaa aagaaaaaat gctttatagc aaaatgaaag agaacatgaa atgcttcttt |
| 1201 | ctcagtttat tggttgaatg tgtatctatt tgagtctgga aataactaat gtgtttgata |
| 1261 | attagtttag tttgtggctt catggaaact ccctgtaaac taaaagcttc agggttatgt |
| 1321 | ctatgttcat tctatagaag aaatgcaaac tatcactgta ttttaatatt tgttattctc |
| 1381 | tcatgaatag aaatttatgt agaagcaaac aaaatacttt tacccactta aaaagagaat |
| 1441 | ataacatttt atgtcactat aatcttttgt tttttaagtt agtgtatatt ttgttgtgat |
| 1501 | tatctttttg tggtgtgaat aaatctttta tcttgaatgt aataagaatt tggtggtgtc |
| 1561 | aattgcttat ttgttttccc acggttgtcc agcaattaat aaaacataac cttttttact |
| 1621 | gcctaaaaaa aaaaaaaaaa a |

With respect to SEQ ID NO:1, the OPNa coding region includes nucleotides 166 to 1110; the exon 4 coding region is indicated in bold and includes nucleotides 259 to 339; and the exon 4 PDAV (SEQ ID NO:5) coding region is indicated in bold underline and includes nucleotides 274 to 285.

In one embodiment of the present invention, OPNa comprises the amino acid sequence of SEQ ID NO:2 (GenBank Accession No. NP_{—}001035147, which is hereby incorporated by reference in its entirety), as follows:

| | |
|---|---|
| 1 | MRIAVICFCL LGITCAIPVK QADSGSSEEK QLYNKYPDAV ATWLNPDPSQ KQNLLAPQNA |
| 61 | VSSEETNDFK QETLPSKSNE SHDHMDDMDD EDDDDHVDSQ DSIDSNDSDD VDDTDDSHQS |
| 121 | DESHHSDESD ELVTDFPTDL PATEVFTPVV PTVDTYDGRG DSVVYGLRSK SKKFRRPDIQ |
| 181 | YPDATDEDIT SHMESEELNG AYKAIPVAQD LNAPSDWDSR GKDSYETSQL DDQSAETHSH |
| 241 | KQSRLYKRKA NDESNEHSDV IDSQELSKVS REFHSHEFHS HEDMLVVDPK SKEEDKHLKF |
| 301 | RISHELDSAS SEVN |

With respect to SEQ ID NO:2, the mature peptide includes amino acid residues 17 to 314, and the amino acid sequence of exon 4 is indicated in bold.

In one embodiment according to the present invention, OPNa exon 4 comprises the amino acid sequence of LYNKYPDAVATWLNPDPSQKQNLLAPQ (SEQ ID NO:3).

In one embodiment according to the present invention, the OPNa inhibitor is an exon-4 specific antibody or binding portion thereof. An antibody of the present invention encompasses any immunoglobulin molecule that specifically binds to an epitope of OPNa. In one embodiment, an antibody of the present invention also encompasses any immunoglobulin molecule that specifically binds to an epitope of OPNa exon 4. As used herein, “epitope” refers to a region of the OPNa exon 4 that is recognized by the isolated antibody and involved in mediating the binding interaction between OPNa exon 4 and its cell-surface receptor, or involved in mediating the downstream molecular signaling pathway triggered by the OPNa exon 4-cell-surface receptor binding interaction.

In one embodiment, the OPNa inhibitor is an antibody or binding fragment thereof that recognizes exon 4 comprising the amino acid sequence LYNKYPDAVATWLNPDPSQKQNLLAPQ (SEQ ID NO:3).

In another embodiment, the OPNa inhibitor is an antibody or binding fragment thereof that recognizes at least a portion of OPNa exon 4. For instance, in one embodiment, the OPNa inhibitor is an antibody or binding fragment thereof that recognizes at least a portion of OPNa exon 4 comprising the amino acid sequence of PDAV (SEQ ID NO:5). In yet another embodiment, the OPNa inhibitor is an antibody or binding fragment thereof that recognizes at least a portion of OPNa exon 4 comprising the amino acid sequence of KYPDAVATWLNPDPSQK (SEQ ID NO:4).

As used herein, the term “antibody” is meant to include intact immunoglobulins derived from natural sources or from recombinant sources, as well as immunoreactive portions (i.e. antigen binding portions) of intact immunoglobulins. The antibodies of the present invention may exist in a variety of forms including, for example, polyclonal antibodies, monoclonal antibodies, intracellular antibodies, antibody fragments (e.g. Fv, Fab and F(ab)2), as well as single chain antibodies (scFv), chimeric antibodies and humanized antibodies (Ed Harlow and David Lane, USING ANTIBODIES: A LABORATORY MANUAL (Cold Spring Harbor Laboratory Press, 1999); Houston et al., “Protein Engineering of Antibody Binding Sites: Recovery of Specific Activity in an Anti-Digoxin Single-Chain Fv Analogue Produced in Escherichia coli,” Proc Natl Acad Sci USA 85:5879-5883 (1988); Bird et al, “Single-Chain Antigen-Binding Proteins,” Science 242:423-426 (1988), which are hereby incorporated by reference in their entirety).

Methods for monoclonal antibody production may be carried out using techniques well-known in the art (MONOCLONAL ANTIBODIES—PRODUCTION, ENGINEERING AND CLINICAL APPLICATIONS (Mary A. Ritter and Heather M. Ladyman eds., 1995), which is hereby incorporated by reference in its entirety). Generally, the process involves obtaining immune cells (lymphocytes) from the spleen of a mammal which has been previously immunized with the antigen of interest (i.e., an epitope of OPNa exon 4) either in vivo or in vitro.

The antibody-secreting lymphocytes are then fused with myeloma cells or transformed cells, which are capable of replicating indefinitely in cell culture, thereby producing an immortal, immunoglobulin-secreting cell line. Fusion with mammalian myeloma cells or other fusion partners capable of replicating indefinitely in cell culture is achieved by standard and well-known techniques, for example, by using polyethylene glycol (PEG) or other fusing agents (Milstein and Kohler, “Derivation of Specific Antibody-Producing Tissue Culture and Tumor Lines by Cell Fusion,” Eur J Immunol 6:511 (1976), which is hereby incorporated by reference in its entirety). The immortal cell line, which is preferably murine, but may also be derived from cells of other mammalian species, is selected to be deficient in enzymes necessary for the utilization of certain nutrients, to be capable of rapid growth, and have good fusion capability. The resulting fused cells, or hybridomas, are cultured, and the resulting colonies screened for the production of the desired monoclonal antibodies. Colonies producing such antibodies are cloned and grown either in vivo or in vitro to produce large quantities of antibody.

Alternatively, monoclonal antibodies can be made using recombinant DNA methods as described in U.S. Pat. No. 4,816,567 to Cabilly et al, which is hereby incorporated by reference in its entirety. The polynucleotides encoding a monoclonal antibody are isolated from mature B-cells or hybridoma cells, for example, by RT-PCR using oligonucleotide primers that specifically amplify the genes encoding the heavy and light chains of the antibody. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, generate monoclonal antibodies. Alternatively, recombinant monoclonal antibodies or fragments thereof of the desired species can be isolated from phage display libraries (McCafferty et al., “Phage Antibodies: Filamentous Phage Displaying Antibody Variable Domains,” Nature 348:552-554 (1990); Clackson et al., “Making Antibody Fragments using Phage Display Libraries,” Nature 352:624-628 (1991); and Marks et al., “By-Passing Immunization. Human Antibodies from V-Gene Libraries Displayed on Phage,” J. Mol. Biol. 222:581-597 (1991), which are hereby incorporated by reference in their entirety).

The polynucleotide(s) encoding a monoclonal antibody can further be modified using recombinant DNA technology to generate alternative antibodies. For example, the constant domains of the light and heavy chains of a mouse monoclonal antibody can be substituted for those regions of a human antibody to generate a chimeric antibody. Alternatively, the constant domains of the light and heavy chains of a mouse monoclonal antibody can be substituted for a non-immunoglobulin polypeptide to generate a fusion antibody. In other embodiments, the constant regions are truncated or removed to generate the desired antibody fragment of a monoclonal antibody. Furthermore, site-directed or high-density mutagenesis of the variable region can be used to optimize specificity and affinity of a monoclonal antibody.

The monoclonal antibody of the present invention can be a humanized antibody. Humanized antibodies contain minimal sequences from non-human (e.g., murine) antibodies within the variable regions. Such antibodies are used therapeutically to reduce antigenicity and human anti-mouse antibody responses when administered to a human subject. In practice, humanized antibodies are typically human antibodies with minimum to no non-human sequences.

An antibody can be humanized by substituting the complementarity determining region (CDR) of a human antibody with that of a non-human antibody (e.g., mouse, rat, rabbit, hamster, etc.) having the desired specificity, affinity, and capability (Jones et al., “Replacing the Complementarity-Determining Regions in a Human Antibody With Those From a Mouse,” Nature 321:522-525 (1986); Riechmann et al., “Reshaping Human Antibodies for Therapy,” Nature 332:323-327 (1988); Verhoeyen et al., “Reshaping Human Antibodies: Grafting an Antilysozyme Activity,” Science 239:1534-1536 (1988), which are hereby incorporated by reference in their entirety). The humanized antibody can be further modified by the substitution of additional residues either in the Fv framework region and/or within the replaced non-human residues to refine and optimize antibody specificity, affinity, and/or capability.

Human antibodies can be produced using various techniques known in the art. For example, immortalized human B lymphocytes immunized in vitro or isolated from an immunized individual that produce an antibody directed against a target antigen can be generated (see e.g., Reisfeld et al., MONOCLONAL ANTIBODIES AND CANCER THERAPY 77 (Alan R. Liss ed., 1985) and U.S. Pat. No. 5,750,373 to Garrard, which are hereby incorporated by reference in their entirety). Also, human antibodies can be selected from a phage library that expresses human antibodies (Vaughan et al., “Human Antibodies with Sub-Nanomolar Affinities Isolated from a Large Non-immunized Phage Display Library,” Nature Biotechnology, 14:309-314 (1996); Sheets et al., “Efficient Construction of a Large Nonimmune Phage Antibody Library: The Production of High-Affinity Human Single-Chain Antibodies to Protein Antigens,” Proc. Natl. Acad. Sci. U.S.A. 95:6157-6162 (1998); Hoogenboom et al., “By-passing Immunisation. Human Antibodies From Synthetic Repertoires of Germline VH Gene Segments Rearranged In Vitro,” J Mol Biol 227:381-8 (1992); Marks et al., “By-passing Immunization. Human Antibodies from V-gene Libraries Displayed on Phage,” J Mol Biol 222:581-97 (1991), which are hereby incorporated by reference in their entirety). Human antibodies can also be made in transgenic mice containing human immunoglobulin loci that are capable upon immunization of producing the full repertoire of human antibodies in the absence of endogenous immunoglobulin production. This approach is described in U.S. Pat. No. 5,545,807 to Surani et al.; U.S. Pat. No. 5,545,806 to Lonberg et al.; U.S. Pat. No. 5,569,825 to Lonberg et al.; U.S. Pat. No. 5,625,126 to Lonberg et al.; U.S. Pat. No. 5,633,425 to Lonberg et al.; and U.S. Pat. No. 5,661,016 to Lonberg et al., which are hereby incorporated by reference in their entirety.

Procedures for raising polyclonal antibodies are also well known. Typically, such antibodies can be raised by administering the peptide or polypeptide containing the epitope of interest subcutaneously to New Zealand white rabbits which have been bled to obtain pre-immune serum. The antigens can be injected in combination with an adjuvant. The rabbits are bled approximately every two weeks after the first injection and periodically boosted with the same antigen three times every six weeks. Polyclonal antibodies are recovered from the serum by affinity chromatography using the corresponding antigen to capture the antibody. This and other procedures for raising polyclonal antibodies are disclosed in Ed Harlow and David Lane, USING ANTIBODIES: A LABORATORY MANUAL (Cold Spring Harbor Laboratory Press, 1988), which is hereby incorporated by reference in its entirety.

In addition to whole antibodies, the present invention encompasses binding portions of such antibodies. Such binding portions include the monovalent Fab fragments, Fv fragments (e.g., single-chain antibody, scFv), single variable V_{H} and V_{L} domains, and the bivalent F(ab′)₂ fragments, Bis-scFv, diabodies, triabodies, minibodies, etc. These antibody fragments can be made by conventional procedures, such as proteolytic fragmentation procedures, as described in James Goding, MONOCLONAL ANTIBODIES: PRINCIPLES AND PRACTICE 98-118 (Academic Press, 1983) and Ed Harlow and David Lane, ANTIBODIES: A LABORATORY MANUAL (Cold Spring Harbor Laboratory, 1988), which are hereby incorporated by reference in their entirety, or other methods known in the art.

The present invention also encompasses the use of bispecific humanized antibodies or bispecific antigen-binding fragments (e.g., F(ab′)₂) which have specificity for OPNa exon 4 and a molecule expressed on a target cell (e.g., cell surface receptor for OPNa). Techniques for making bispecific antibodies are common in the art (Brennan et al., “Preparation of Bispecific Antibodies by Chemical Recombination of Monoclonal Immunoglobulin G1 Fragments,” Science 229:81-3 (1985); Suresh et al, “Bispecific Monoclonal Antibodies From Hybrid Hybridomas,” Methods in Enzymol. 121:210-28 (1986); Traunecker et al., “Bispecific Single Chain Molecules (Janusins) Target Cytotoxic Lymphocytes on HIV Infected Cells,” EMBO J. 10:3655-3659 (1991); Shalaby et al., “Development of Humanized Bispecific Antibodies Reactive with Cytotoxic Lymphocytes and Tumor Cells Overexpressing the HER2 Protooncogene,” J. Exp. Med. 175:217-225 (1992); Kostelny et al, “Formation of a Bispecific Antibody by the Use of Leucine Zippers,” J. Immunol. 148: 1547-1553 (1992); Gruber et al., “Efficient Tumor Cell Lysis Mediated by a Bispecific Single Chain Antibody Expressed in Escherichia coli,” J. Immunol. 152:5368-74 (1994); and U.S. Pat. No. 5,731,168 to Carter et al., which are hereby incorporated by reference in their entirety). Generally, bispecific antibodies are secreted by triomas (i.e., lymphoma cells fuse to a hybridoma) and hybrid hybridomas. The supernatants of triomas and hybrid hybridomas can be assayed for bispecific antibody production using a suitable assay (e.g., ELISA), and bispecific antibodies can be purified using conventional methods. These antibodies can then be humanized according to methods known in the art. Humanized bispecific antibodies or a bivalent antigen-binding fragment of the bispecific antibody having binding specificity for OPNa exon 4 and an antigen expressed on a target cell, provides a cell-specific targeting approach.

It may further be desirable, especially in the case of antibody fragments, to modify the antibody in order to increase its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

The present invention also encompasses the nucleic acid molecules that encode the OPNa exon 4-specific antibodies of the invention. The nucleic acids may be present in whole cells, in a cell lysate, or in a partially purified or substantially pure form (i.e., purified away from other cellular components or other contaminants).

Nucleic acids encoding the antibodies of the present invention can be obtained using standard molecular biology techniques. For antibodies expressed by hybridomas, cDNAs encoding the light and heavy chains of the antibody made by the hybridoma can be obtained by standard PCR amplification or cDNA cloning techniques. For antibodies obtained from an immunoglobulin gene library (e.g., using phage display techniques), the nucleic acid encoding the antibody can be recovered from the library.

Preferred nucleic acid molecules of the invention are those encoding the V_{H} and V_{L} sequences of OPNa exon-4 monoclonal antibodies. Once DNA or cDNA fragments encoding V_{H} and V_{L} segments are obtained, these DNA fragments can be further manipulated by standard recombinant DNA techniques, for example to convert the variable region genes to full-length antibody chain genes, to Fab fragment genes, or to a scFv gene. In these manipulations, a V_{L}- or V_{H}-encoding DNA fragment is operatively linked to another DNA fragment encoding another protein, such as an antibody constant region, or a flexible linker. The isolated DNA encoding the V_{H} region can be converted to a full-length heavy chain gene by operatively linking the V_{H}-encoding DNA to another DNA molecule encoding heavy chain constant regions (CH1, CH2 and CH3). The sequences of human heavy chain constant region genes are known in the art (see e.g., Kabat et al., Sequences of Proteins of Immunological Interest, 5^{th} ed., U.S. Department of Health and Human Services, NIH Publication No. 91-3242 (1991), which is hereby incorporated by reference in its entirety) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. For a Fab fragment heavy chain gene, the V_{H}-encoding DNA can be operatively linked to another DNA molecule encoding only the heavy chain CH1 constant region.

The isolated DNA encoding the V_{L} region can be converted to a full-length light chain gene (as well as a Fab light chain gene) by operatively linking the V_{L}-encoding DNA to another DNA molecule encoding the light chain constant region. The sequences of human light chain constant region genes are known in the art (see e.g., Kabat et al., Sequences of Proteins of Immunological Interest, 5^{th} ed., U.S. Department of Health and Human Services, NIH Publication No. 91-3242 (1991), which is hereby incorporated by reference in its entirety) and DNA fragments encompassing these regions can be obtained by standard PCR amplification. The light chain constant region can be a kappa or lambda constant region, but most preferably is a kappa constant region.

To create a scFv gene, the V_{H}- and V_{L}-encoding DNA fragments are operatively linked to another fragment encoding a flexible linker such that the V_{H} and V_{L} sequences can be expressed as a contiguous single-chain protein, with the V_{H} and V_{L} regions joined by the flexible linker (see e.g., Bird et al., “Single Chain Antigen-Binding Proteins,” Science 242:423-426 (1988); Huston et al., “Protein Engineering of Antibody Binding Sites: Recovery of Specific Activity in an Anti-Digoxin Single-Chain Fv Analogue Produced in Escherichia coli,” Proc. Natl. Acad. Sci. USA 85:5879-5883 (1988); McCafferty et al., “Phage Antibodies: Filamentous Phage Displaying Antibody Variable Domains,” Nature 348:552-554 (1990), which are hereby incorporated by reference in their entirety).

Antibody mimics are also suitable inhibitors for use in accordance with the present invention. A number of antibody mimics are known in the art including, without limitation, those known as monobodies, which are derived from the tenth human fibronectin type III domain (¹⁰Fn3) (Koide et al., “The Fibronectin Type III Domain as a Scaffold for Novel Binding Proteins,” J. Mol. Biol. 284:1141-1151 (1998); Koide et al., “Probing Protein Conformational Changes in Living Cells by Using Designer Binding Proteins: Application to the Estrogen Receptor,” Proc. Natl. Acad. Sci. USA 99:1253-1258 (2002), each of which is hereby incorporated by reference in its entirety); and those known as affibodies, which are derived from the stable alpha-helical bacterial receptor domain Z of staphylococcal protein A (Nord et al., “Binding Proteins Selected from Combinatorial Libraries of an alpha-helical Bacterial Receptor Domain,” Nature Biotechnol. 15(8):772-777 (1997), which is hereby incorporated by reference in its entirety).

In another embodiment of the present invention, the OPNa inhibitor is a peptide mimic of OPNa exon 4 or a fragment thereof.

Suitable peptide mimics according to the present invention include those having the formula (R₁)ₓ-R₂-(R₃)_{y}, where R₁ is an amino acid sequence comprising from 1 to 30 amino acids, where each amino acid is independently selected from the group consisting of naturally occurring amino acids and amino acid analogs; R₂ is PDAV (SEQ ID NO:5); R₃ comprises is an amino acid sequence comprising from 1 to 30 amino acids, where each amino acid is independently selected from the group consisting of naturally occurring amino acids and amino acid analogs; and x and y are independently selected and equal to zero or one.

In one embodiment, R₁ is an amino acid sequence comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids. According to one embodiment of the present invention, R₁ is selected from the group consisting of Y, KY, NKY, YNKY (SEQ ID NO:6), and LYNKY (SEQ ID NO:7).

In one embodiment, R₃ is an amino acid sequence comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids. According to one embodiment of the present invention, R₃ is selected from the group consisting of A, AT, ATW, ATWL (SEQ ID NO:8), ATWLN (SEQ ID NO:9), ATWLNP (SEQ ID NO:10), ATWLNPD (SEQ ID NO:11), ATWLNPDP (SEQ ID NO:12), ATWLNPDPS (SEQ ID NO:13), ATWLNPDPSQ (SEQ ID NO:14), ATWLNPDPSQK (SEQ ID NO:15), ATWLNPDPSQKQ (SEQ ID NO:16), ATWLNPDPSQKQN (SEQ ID NO:17), ATWLNPDPSQKQNL (SEQ ID NO:18), ATWLNPDPSQKQNLL (SEQ ID NO:19), ATWLNPDPSQKQNLLA (SEQ ID NO:20), ATWLNPDPSQKQNLLAP (SEQ ID NO:21), and ATWLNPDPSQKQNLLAPQ (SEQ ID NO:22).

In one embodiment of the present invention, the peptide mimic of OPNa exon 4 comprises the amino acid sequence selected from the group consisting of PDAV (SEQ ID NO:5); KYPDAVATWLNPDPSQK (SEQ ID NO:4); and LYNKYPDAVATWLNPDPSQKQNLLAPQ (SEQ ID NO:3). In another embodiment, the peptide mimic according to the present invention comprises an amino acid sequence having at least about 80%, 85%, 90%, 95%, 96%, 97%, 98%, or 99% sequence identity to the amino acid sequence of KYPDAVATWLNPDPSQK (SEQ ID NO:4) and/or LYNKYPDAVATWLNPDPSQKQNLLAPQ (SEQ ID NO:3).

In one embodiment of the present invention, x and y are 1. In another embodiment of the present invention, x is 1 and y is 0. In yet another embodiment of the present invention, x is 0 and y is 1. In yet another embodiment of the present invention x and y are 0.

In yet another embodiment of the present invention, the OPNa inhibitor is a nucleic acid aptamer that specifically binds to OPNa exon 4 or a fragment thereof.

In one embodiment of the present invention, the nucleic acid aptamer binds to at least a fragment of OPNa exon 4 of SEQ ID NO:3. In one embodiment, the nucleic acid aptamer binds to at least a fragment of OPNa exon 4 comprising the amino acid sequence PDAV (SEQ ID NO:5).

Nucleic acid aptamers are single-stranded, partially single-stranded, partially double-stranded, or double-stranded nucleotide sequences, advantageously a replicatable nucleotide sequence, capable of specifically recognizing a selected non-oligonucleotide molecule or group of molecules by a mechanism other than Watson-Crick base pairing or triplex formation. Aptamers include, without limitation, defined sequence segments and sequences comprising nucleotides, ribonucleotides, deoxyribonucleotides, nucleotide analogs, modified nucleotides, and nucleotides comprising backbone modifications, branchpoints, and non-nucleotide residues, groups, or bridges. Nucleic acid aptamers include partially and fully single-stranded and double-stranded nucleotide molecules and sequences; synthetic RNA, DNA, and chimeric nucleotides; hybrids; duplexes; heteroduplexes; and any ribonucleotide, deoxyribonucleotide, or chimeric counterpart thereof and/or corresponding complementary sequence, promoter, or primer-annealing sequence needed to amplify, transcribe, or replicate all or part of the aptamer molecule or sequence.

In yet another embodiment of the present invention, the OPNa inhibitor is a peptide inhibitor that binds to OPNa exon 4 or a portion thereof. Suitable inhibitory peptides of the present invention are short peptides based on the sequence of OPNa that exhibit inhibition of OPNa binding to and direct biological antagonist activity. The amino acid sequence of human OPNa exon 4 from which inhibitory peptides are derived is the amino acid sequence of SEQ ID NO: 3.

In one embodiment of the present invention, the peptide inhibitor binds to at least a fragment of OPNa exon 4 of SEQ ID NO:3. In one embodiment, the peptide inhibitor binds to at least a fragment of OPNa exon 4 comprising the amino acid sequence PDAV (SEQ ID NO:5).

Inhibitory peptides or peptide mimics according to the present invention may be chemically synthesized using known peptide synthesis methodology or may be prepared and purified using recombinant technology. Such peptides are usually at least about 4 amino acids in length, but can be anywhere from 4 to 100 amino acids in length. Such peptides may be identified without undue experimentation using well known techniques. Techniques for screening peptide libraries for peptides that are capable of specifically binding to a polypeptide target, in this case OPNa exon 4, are well known in the art (see e.g., U.S. Pat. No. 5,556,762 to Pinilla et al.; U.S. Pat. No. 5,750,373 to Garrard et al.; U.S. Pat. No. 4,708,871 to Geysen; U.S. Pat. No. 4,833,092 to Geysen; U.S. Pat. No. 5,223,409 to Ladner et al.; U.S. Pat. No. 5,403,484 to Ladner et al.; U.S. Pat. No. 5,571,689 to Heuckeroth et al.; U.S. Pat. No. 5,663,143 to Ley et al.; and PCT Publication Nos. WO84/03506 to Geysen and WO84/03564 to Geysen, which are hereby incorporated by reference in their entirety).

In one embodiment of the present invention, the therapeutic further comprises a cancer therapeutic. Cancer therapeutics according to the present invention include, inter alia, chemotherapeutics. Suitable chemotherapeutics according to the present invention include those selected from the group consisting of alkylating agents, antimetabolites, anthracyclines, antitumor antibiotics, platinum-based chemotherapeutics, plant alkaloids, and combinations thereof. In one embodiment of the present invention, the chemotherapeutic is a platinum-based chemotherapeutic. In one particular embodiment of the present invention the chemotherapeutic is cisplatin.

Suitable cancer therapeutics according to the present invention also include those selected from the group consisting of oxaliplatin, cyclophosphamide, ifosfamide, thiotepa, melphalan, busulfan, nimustine, ranimustine, dacarbazine, procarbazine, temozolomide, cisplatin, carboplatin, nedaplatin, methotrexate, pemetrexed, fluorouracil, tegaful/uracil, doxifluridine, tegaful/gimeracil/oteracil, capecitabine, cytarabine, enocitabine, gemcitabine, 6-mercaptopurine, fuludarabin, pentostatin, cladribine, hydroxyurea, doxorubicin, epirubicin, daunorubicin, idarubicine, pirarubicin, mitoxantrone, amurubicin, actinomycin D, bleomycine, pepleomycin, mytomycin C, aclarubicin, zinostatin, vincristine, vindesine, vinblastine, vinorelbine, paclitaxel, docetaxel, irinotecan, irinotecan active metabolite (SN-38), nogitecan (topotecan), etoposide, prednisolone, dexamethasone, tamoxifen, toremifene, medroxyprogesterone, anastrozole, exemestane, letrozole, rituximab, imatinib, gefitinib, gemtuzumab ozogamicin, bortezomib, erlotinib, cetuximab, bevacizumab, sunitinib, sorafenib, dasatinib, panitumumab, asparaginase, tretinoin, arsenic trioxide, salts thereof, active metabolites thereof, and combinations thereof.

Another aspect of the present invention relates to a method of inhibiting tumor growth and/or metastasis in a subject. This method includes selecting a subject having a tumor, where the tumor cells express OPNa exon 4 and administering to the selected subject an OPNa inhibitor according to the present invention.

In one embodiment, an OPNa inhibitor and a cancer therapeutic are administered. In another embodiment, the OPNa inhibitor is administered in the form of a therapeutic according to the present invention that comprises an OPNa inhibitor. In another embodiment, the OPNa inhibitor is administered in the form of a therapeutic according to the present invention that comprises an OPNa inhibitor and a cancer therapeutic. In one embodiment, the OPNa inhibitor blocks activity of extracellular OPNa exon 4 and is selected from the group consisting of (i) an exon-4 specific antibody or binding portion thereof; (ii) a peptide mimic of OPNa exon 4 or a fragment thereof; (iii) a nucleic acid aptamer that specifically binds to OPNa exon 4 or a fragment thereof; and (iv) a peptide inhibitor that binds to OPNa exon 4 or a fragment thereof.

In certain embodiments, the selected subject has a tumor that is capable of metastasizing, but has not yet metastasized to tissues or organs elsewhere in the body and metastasis is prevented or inhibited in the subject. In certain embodiments, the selected subject has a tumor that has metastasized to tissues or organs elsewhere in the body and further metastasis is prevented or inhibited.

Inhibition of tumor growth according to the present invention includes, for example, inhibition of the rate of tumor growth, as well as inhibition or slowing of increase in tumor size, and inhibition of tumor cell growth. Inhibiting tumor cell growth is meant to include, for example, a decrease of the number of tumor cells entering the cell cycle, tumor cell death or the decrease of tumor cell metastasis. Metastasis is the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream or lymphatics, and stopping at a distant site. After the tumor cells come to rest at another site, they re-penetrate through the blood vessels or lymphatic walls, continue to multiply, and eventually another tumor is formed. At the new site, the cells establish a blood supply and can grow to form a life-threatening mass.

Another aspect of the present invention relates to a method of treating a subject having chemotherapeutic resistance. This method includes selecting a subject having chemotherapeutic resistance and administering to the selected subject an OPNa inhibitor, where the OPNa inhibitor blocks activity of extracellular OPNa exon 4. In one embodiment, an OPNa inhibitor and a cancer therapeutic are administered.

In one embodiment, the OPNa inhibitor is administered in the form of a therapeutic according to the present invention that comprises an OPNa inhibitor. In another embodiment, the OPNa inhibitor is administered in the form of a therapeutic according to the present invention that comprises an OPNa inhibitor and a cancer therapeutic. In one embodiment, the OPNa inhibitor blocks activity of extracellular OPNa exon 4 and is selected from the group consisting of (i) an exon-4 specific antibody or binding portion thereof; (ii) a peptide mimic of OPNa exon 4 or a fragment thereof; (iii) a nucleic acid aptamer that specifically binds to OPNa exon 4 or a fragment thereof; and (iv) a peptide inhibitor that binds to OPNa exon 4 or a fragment thereof.

As will be understood, one significant problem of chemotherapy is that tumors can develop resistance to drugs. For example, a drug may be highly effective when it is first introduced to the patient, killing tumor cells and reducing the size of the tumor. However, the tumor may regrow after a period of time, and the same drug is not effective at killing the regrown tumor cells. Thus, treatment of the subject includes, for example, administering an amount of a therapeutic according to the present invention, in conjunction or succession with a cancer therapeutic, which is sufficient to increase the susceptibility or sensitivity of cancer cells to the cancer therapeutic, or is sufficient to enhance the cytotoxicity of the cancer therapeutic, thereby treating the subject having cancer therapeutic or chemotherapeutic resistance.

In accordance with the present invention, an increase in sensitivity of a tumor or neoplastic cell to a cancer therapeutic or chemotherapeutic (or reduction in chemotherapeutic resistance) is indicated, for example, by reduction in viability or growth rate of the cell in response to the chemotherapeutic agent. For instance, to determine if a tumor cell is growth inhibited, the growth rate of the cell in the presence or absence of the cancer therapeutic or chemotherapeutic can be determined by established methods in the art. The tumor cell is not growth inhibited by the chemotherapeutic agent if the growth rate is not significantly different with or without the chemotherapeutic. The increase in tumor cell sensitivity (or reduction in chemotherapeutic resistance) may also be demonstrated by a reduction of the symptoms caused by the tumor cells.

Yet a further aspect of the present invention relates to a method for increasing tumor cell sensitivity to a cancer therapeutic in a subject. The method includes selecting a subject in need of increased tumor cell sensitivity to a cancer therapeutic; providing an OPNa inhibitor; providing the cancer therapeutic; and administering to the subject the OPNa inhibitor and the cancer therapeutic under conditions effective to increase tumor cell sensitivity to the cancer therapeutic in the selected subject as compared to administration of the cancer therapeutic alone.

The OPNa inhibitor and the cancer therapeutic of methods according to the present invention may be administered simultaneously or in any sequence. In one embodiment of the present invention the OPNa inhibitor is administered to the subject before the cancer therapeutic is administered. In another embodiment according to the present invention the OPNa inhibitor is administered to the subject after the cancer therapeutic is administered.

In certain embodiments, the selected subject according to the present invention has a tumor that displays cancer therapeutic or chemotherapeutic resistance. As noted above, in accordance with the present invention, sensitivity of a tumor or neoplastic cell to a chemotherapeutic is indicated by reduction in viability or growth rate of the cell in response to the chemotherapeutic agent. An effective amount of a therapeutic according to the present invention administered to a subject, in conjunction with a cancer therapeutic or chemotherapeutic, which is sufficient to increase the susceptibility of cancer cells to the cancer therapeutic or chemotherapeutic or is sufficient to enhance the cytotoxicity of the cancer therapeutic or chemotherapeutic.

The subject according to the present invention is preferably a mammal, particularly a dog, cat, sheep, goat, cattle, horse, pig, human, or non-human primate. In one embodiment, the subject is a human.

The selected subject according to certain aspects of the present invention has a tumor. The tumor may be of any origin so long as its metastasis and/or cancer therapeutic or chemotherapeutic resistance is mediated by OPNa. In one embodiment the subject has lung cancer. In one particular embodiment, the subject has NSCLC.

Suitable OPNa inhibitors according to the present invention include those described above, as well as nucleic acid inhibitors. Exemplary nucleic acid inhibitors of OPNa include antisense RNAs or RNAi, such as short interfering RNAs (siRNA), short hairpin RNAs (shRNA), and microRNAs.

The use of antisense methods to inhibit the in vivo translation of genes and subsequent protein expression is well known in the art (e.g., U.S. Pat. No. 7,425,544 to Dobie et al.; U.S. Pat. No. 7,307,069 to Karras et al.; U.S. Pat. No. 7,288,530 to Bennett et al.; U.S. Pat. No. 7,179,796 to Cowsert et al., which are hereby incorporated by reference in their entirety). Antisense nucleic acids are nucleic acid molecules (e.g., molecules containing DNA nucleotides, RNA nucleotides, or modifications (e.g., modification that increase the stability of the molecule, such as 2′-O-alkyl (e.g., methyl) substituted nucleotides) or combinations thereof) that are complementary to, or that hybridize to, at least a portion of a specific nucleic acid molecule, such as an mRNA molecule (see e.g., Weintraub, H. M., “Antisense DNA and RNA,” Scientific Am. 262:40-46 (1990), which is hereby incorporated by reference in its entirety). The antisense nucleic acid molecule hybridizes to its corresponding target nucleic acid molecule, such as the OPNa mRNA, to form a double-stranded molecule, which interferes with translation of the mRNA, as the cell will not translate a double-stranded mRNA.

The nucleotide sequence of OPNa, which can be used for designing antisense and other nucleic acid inhibitory molecules is provided above as SEQ ID NO:1.

Antisense nucleic acids used in the methods of the present invention are typically at least 10-12 nucleotides in length, for example, at least 15, 20, 25, 50, 75, or 100 nucleotides in length. The antisense nucleic acid can also be as long as the target nucleic acid with which it is intended to form an inhibitory duplex. Antisense nucleic acids can be introduced into cells as antisense oligonucleotides, or can be produced in a cell in which a nucleic acid encoding the antisense nucleic acid has been introduced, for example, using gene therapy methods.

siRNAs are double stranded synthetic RNA molecules approximately 20-25 nucleotides in length with short 2-3 nucleotide 3′ overhangs on both ends. The double stranded siRNA molecule represents the sense and anti-sense strand of a portion of the target mRNA molecule, in this case a portion of the OPNa nucleotide sequence. siRNA molecules are typically designed to target a region of the mRNA target approximately 50-100 nucleotides downstream from the start codon. Upon introduction into a cell, the siRNA complex triggers the endogenous RNA interference (RNAi) pathway, resulting in the cleavage and degradation of the target mRNA molecule. siRNA molecules that effectively interfere with OPNa expression have been developed (see Goparaju et al. “Functional Heterogeneity of Osteopontin Isoforms in Non-small Cell Lung Cancer,” J Thorac Oncol 5:1516-23 (2010); Blasberg et al., “Lung Cancer Osteopontin Isoforms Exhibit Angiogenic Functional Heterogeneity,” The Journal of Thoracic and Cardiovascular Surgery 139:1587-93 (2010); and Zhao et al., “Osteopontin as a Potential Biomarker of Proliferation and Invasiveness for Lung Cancer,” J Cancer Res Clin Oncol 137:1061-70 (2011), which are hereby incorporated by reference in their entirety) and are suitable for use in the present invention. Various improvements of siRNA compositions, such as the incorporation of modified nucleosides or motifs into one or both strands of the siRNA molecule to enhance stability, specificity, and efficacy, have been described and are suitable for use in accordance with this aspect of the invention (see e.g., WO2004/015107 to Giese et al.; WO2003/070918 to McSwiggen et al.; WO1998/39352 to Imanishi et al.; U.S. Patent Application Publication No. 2002/0068708 to Jesper et al.; U.S. Patent Application Publication No. 2002/0147332 to Kaneko et al; U.S. Patent Application Publication No. 2008/0119427 to Bhat et al., which are hereby incorporated by reference in their entirety).

Short or small hairpin RNA molecules are similar to siRNA molecules in function, but comprise longer RNA sequences that make a tight hairpin turn. shRNA is cleaved by cellular machinery into siRNA and gene expression is silenced via the cellular RNA interference pathway.

In accordance with the methods of the present invention, the mode of administering the therapeutic agent of the present invention, including the use of suitable delivery vehicles, to a subject will vary depending on the type of therapeutic agent (e.g., nucleic acid molecule, peptide inhibitor, antibody, peptide mimic, or small molecule).

In one embodiment, nucleic acid molecules encoding exon-4 specific antibodies or antibody binding fragments, nucleic acid molecules encoding peptide mimics of OPNa exon 4 or a fragment thereof, inhibitory OPNa exon 4 nucleic acid molecules (i.e., nucleic acid aptamer that specifically binds to OPNa exon 4), or nucleic acid molecules encoding peptide inhibitors that bind to OPNa exon 4 or a fragment thereof may be incorporated into a gene therapy vector to facilitate delivery.

In a preferred embodiment, the gene therapy vector carrying or encoding the inhibitory molecule is an expression vector derived from a virus. Suitable viral vectors include, without limitation, adenovirus, adeno-associated virus, retrovirus, lentivirus, or herpes virus.

Adenoviral viral vector gene delivery vehicles can be readily prepared and utilized as described in Berkner, “Development of Adenovirus Vectors for the Expression of Heterologous Genes,” Biotechniques 6:616-627 (1988) and Rosenfeld et al., “Adenovirus-Mediated Transfer of a Recombinant Alpha 1-Antitrypsin Gene to the Lung Epithelium In Vivo,” Science 252:431-434 (1991), WO 93/07283 to Curiel et al., WO 93/06223 to Perricaudet et al., and WO 93/07282 to Curiel et al., which are hereby incorporated by reference in their entirety. Adeno-associated viral gene delivery vehicles can be constructed and used to deliver a gene, including a gene encoding an antibody to cells as described in Shi et al., “Therapeutic Expression of an Anti-Death Receptor-5 Single-Chain Fixed Variable Region Prevents Tumor Growth in Mice,” Cancer Res. 66:11946-53 (2006); Fukuchi et al., “Anti-A13 Single-Chain Antibody Delivery via Adeno-Associated Virus for Treatment of Alzheimer's Disease,” Neurobiol. Dis. 23:502-511 (2006); Chatterjee et al., “Dual-Target Inhibition of HIV-1 In Vitro by Means of an Adeno-Associated Virus Antisense Vector,” Science 258:1485-1488 (1992); Ponnazhagan et al., “Suppression of Human Alpha-Globin Gene Expression Mediated by the Recombinant Adeno-Associated Virus 2-Based Antisense Vectors,” J. Exp. Med. 179:733-738 (1994); and Zhou et al., “Adeno-Associated Virus 2-Mediated Transduction and Erythroid Cell-Specific Expression of a Human Beta-Globin Gene,” Gene Ther. 3:223-229 (1996), which are hereby incorporated by reference in their entirety. In vivo use of these vehicles is described in Flotte et al., “Stable In Vivo Expression of the Cystic Fibrosis Transmembrane Conductance Regulator With an Adeno-Associated Virus Vector,” Proc. Nat'l. Acad. Sci. 90:10613-10617 (1993) and Kaplitt et al., “Long-Term Gene Expression and Phenotypic Correction Using Adeno-Associated Virus Vectors in the Mammalian Brain,” Nature Genet. 8:148-153 (1994), which are hereby incorporated by reference in their entirety. Additional types of adenovirus vectors are described in U.S. Pat. No. 6,057,155 to Wickham et al.; U.S. Pat. No. 6,033,908 to Bout et al.; U.S. Pat. No. 6,001,557 to Wilson et al.; U.S. Pat. No. 5,994,132 to Chamberlain et al.; U.S. Pat. No. 5,981,225 to Kochanek et al.; U.S. Pat. No. 5,885,808 to Spooner et al.; and U.S. Pat. No. 5,871,727 to Curiel, which are hereby incorporated by reference in their entirety.

Retroviral vectors which have been modified to form infective transformation systems can also be used to deliver inhibitory nucleic acid molecules or nucleic acid molecules encoding a desired peptide or antibody to a target cell. One such type of retroviral vector is disclosed in U.S. Pat. No. 5,849,586 to Kriegler et al., which is hereby incorporated by reference.

Gene therapy vectors carrying the therapeutic nucleic acid molecule are administered to a subject by, for example, intravenous injection, local administration (U.S. Pat. No. 5,328,470 to Nabel et al., which is hereby incorporated by reference in its entirety) or by stereotactic injection (see e.g., Chen et al. “Gene Therapy for Brain Tumors: Regression of Experimental Gliomas by Adenovirus Mediated Gene Transfer In Vivo,” Proc. Nat'l. Acad. Sci. USA 91:3054-3057 (1994), which is hereby incorporated by reference in its entirety). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system. Gene therapy vectors typically utilize constitutive regulatory elements which are responsive to endogenous transcriptions factors.

Another suitable approach for the delivery of the therapeutic agents of the present invention involves the use of liposome delivery vehicles.

Liposomes are vesicles comprised of one or more concentrically ordered lipid bilayers which encapsulate an aqueous phase. They are normally not leaky, but can become leaky if a hole or pore occurs in the membrane, if the membrane is dissolved or degrades, or if the membrane temperature is increased to the phase transition temperature. Current methods of drug delivery via liposomes require that the liposome carrier ultimately become permeable and release the encapsulated therapeutic agent at the primary target site. This can be accomplished, for example, in a passive manner where the liposome bilayer degrades over time through the action of various agents in the body.

In contrast to passive drug release, active drug release using liposome delivery vehicles can also be achieved. For example, liposome membranes can be constructed to be pH sensitive (see e.g., Wang & Huang, “pH-Sensitive Immunoliposomes Mediate Target-cell-specific Delivery and Controlled Expression of a Foreign Gene in Mouse,” Proc. Nat'l Acad. Sci. USA 84:7851-5 (1987), which is hereby incorporated by reference in its entirety). When liposomes are endocytosed by a target cell, for example, they can be routed to acidic endosomes which will destabilize the liposome and result in drug release. Alternatively, the liposome membrane can be chemically modified such that an enzyme placed as a coating on the membrane slowly destabilizes the liposome.

Different types of liposomes can be prepared using methods known in the art, see e.g., Bangham et al., “Diffusion of Univalent Ions Across the Lamellae of Swollen Phospholipids,” J. Mol. Biol. 13:238-52 (1965); U.S. Pat. No. 5,653,996 to Hsu; U.S. Pat. No. 5,643,599 to Lee et al.; U.S. Pat. No. 5,885,613 to Holland et al.; U.S. Pat. No. 5,631,237 to Dzau & Kaneda; and U.S. Pat. No. 5,059,421 to Loughrey et al., which are hereby incorporated by reference in their entirety.

Yet another approach for delivery of a therapeutic according to the present invention involves the conjugation of the desired OPNa inhibitor of the present invention (e.g., peptide mimic, peptide inhibitor, etc.) to a stabilized polymer to avoid enzymatic degradation of the inhibitory peptide. Conjugated peptides or polypeptides of this type are described in U.S. Pat. No. 5,681,811 to Ekwuribe, which is hereby incorporated by reference in its entirety.

The therapeutic agents of the present invention can be administered via any standard route of administration known in the art, including, but not limited to, parenteral (e.g., intravenous, intraarterial, intramuscular, subcutaneous injection, intrathecal), oral (e.g., dietary), topical, transmucosal, or by inhalation (e.g., intrabronchial, intranasal or oral inhalation, intranasal drops). Typically, parenteral administration is the preferred mode of administration.

Therapeutic agents of the present invention are formulated in accordance with their mode of administration. For oral administration, for example, the therapeutic agents of the present invention are formulated into an inert diluent or an assimilable edible carrier, enclosed in hard or soft shell capsules, compressed into tablets, or incorporated directly into food. Agents of the present invention may also be administered in a time release manner incorporated within such devices as time-release capsules or nanotubes. Such devices afford flexibility relative to time and dosage. For oral therapeutic administration, the agents of the present invention may be incorporated with excipients and used in the form of tablets, capsules, elixirs, suspensions, syrups, and the like. Such compositions and preparations should contain at least 0.1% of the agent, although lower concentrations may be effective and indeed optimal. The percentage of the agent in these compositions may, of course, be varied and may conveniently be between about 2% to about 60% of the weight of the unit. The amount of an agent of the present invention in such therapeutically useful compositions is such that a suitable dosage will be obtained.

Also specifically contemplated are oral dosage forms of the agents of the present invention. The agents may be chemically modified so that oral delivery of the derivative is efficacious. Generally, the chemical modification contemplated is the attachment of at least one moiety to the component molecule itself, where said moiety permits inhibition of proteolysis and uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline (Abuchowski and Davis, “Soluble Polymer-Enzyme Adducts,” In: Enzymes as Drugs, Hocenberg and Roberts, eds., Wiley-Interscience (1981), which is hereby incorporated by reference in their entirety). Other polymers that could be used are poly-1,3-dioxolane and poly-1,3,6-tioxocane. Preferred for pharmaceutical usage, as indicated above, are polyethylene glycol moieties.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, sucrulose, or saccharin. When the dosage unit form is a capsule, it may contain, in addition to materials of the above type, a liquid carrier such as a fatty oil.

The therapeutics of the present invention may also be formulated for parenteral administration. Solutions or suspensions of the agent can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

Pharmaceutical formulations suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

When it is desirable to deliver the agents of the present invention systemically, they may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Intraperitoneal or intrathecal administration of the agents of the present invention can also be achieved using infusion pump devices such as those described by Medtronic, Northridge, Calif. Such devices allow continuous infusion of desired compounds avoiding multiple injections and multiple manipulations.

In addition to the formulations described previously, the therapeutics or agents according to the present invention may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

Effective doses of the therapeutics or agents of the present invention vary depending upon many different factors, including type and stage of disease, mode of administration, target site, physiological state of the patient, other medications or therapies administered, and physical state of the patient relative to other medical complications. Treatment dosages need to be titrated to optimize safety and efficacy.

The therapeutics of the present invention can be administered in a single dose or multiple doses. The dosage can be determined by methods known in the art and can be dependent, for example, upon the individual's age, sensitivity, tolerance and overall well-being. For example, suitable dosages for antibodies can be from about 0.1 mg/kg body weight to about 10.0 mg/kg body weight per treatment.

The therapeutic agents of the present invention can be administered to an individual (e.g., a human) alone or in conjunction with one or more other therapeutic agents of the invention. As described supra, when the therapeutic agent of the present invention is administered to an individual having a tumor to prevent tumor growth and/or metastasis, the therapeutic agent can be administered in conjunction with one or more cancer therapeutic (e.g., chemotherapeutic) agents.

### EXAMPLES

The following examples are provided to illustrate embodiments of the present invention but are by no means intended to limit its scope.

### Example 1

### Targeting Osteopontin Splice Variation to Increase Chemosensitivity and Decrease Malignant Potential in Non-Small Cell Lung Cancer

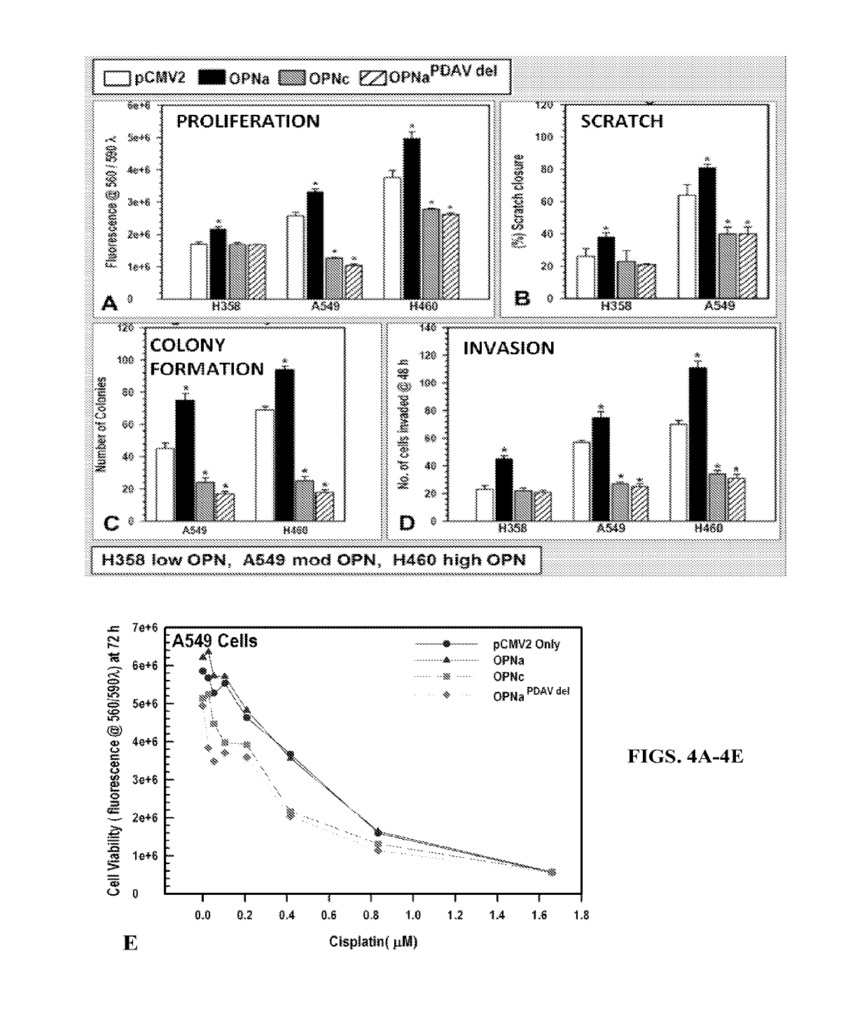

As noted above, it has been demonstrated that the three naturally occurring OPN splice variants are differentially expressed in NSCLC, with the full length isoform, OPNa, being preferentially up-regulated in tumors compared to normal tissue, as shown in FIG. 2 (Goparaju et al. “Functional Heterogeneity of Osteopontin Isoforms in Non-small Cell Lung Cancer,” J Thorac Oncol 5:1516-23 (2010) and Blasberg et al., “Lung Cancer Osteopontin Isoforms Exhibit Angiogenic Functional Heterogeneity,” The Journal of Thoracic and Cardiovascular Surgery 139:1587-93 (2010), which are hereby incorporated by reference in their entirety). It has also been demonstrated that increased OPN expression in NSCLC cell lines is associated with increased malignant behavior in vitro and in vivo and malignant properties can be abrogated with si-RNA directed against OPN (Goparaju et al. “Functional Heterogeneity of Osteopontin Isoforms in Non-small Cell Lung Cancer,” J Thorac Oncol 5:1516-23 (2010); Blasberg et al., “Lung Cancer Osteopontin Isoforms Exhibit Angiogenic Functional Heterogeneity,” The Journal of Thoracic and Cardiovascular Surgery 139:1587-93 (2010); and Zhao et al., “Osteopontin as a Potential Biomarker of Proliferation and Invasiveness for Lung Cancer,” J Cancer Res Clin Oncol 137:1061-70 (2011), which are hereby incorporated by reference in their entirety). The inventors have extensively characterized three NSCLC cell lines with regard to OPN isoform expression and secretion, H358, A549 and H460, which is illustrated in FIG. 3 (Goparaju et al. “Functional Heterogeneity of Osteopontin Isoforms in Non-small Cell Lung Cancer,” J Thorac Oncol 5:1516-23 (2010) and Blasberg et al., “Lung Cancer Osteopontin Isoforms Exhibit Angiogenic Functional Heterogeneity,” The Journal of Thoracic and Cardiovascular Surgery 139:1587-93 (2010), which are hereby incorporated by reference in their entirety). Expression of the full-length OPN in these cell lines parallels the clinical behavior of the cell type and the in vitro functional profile. Specifically, H358 (low OPN) is an adenocarcinoma in-situ cell line, an indolent subtype with low invasive potential, which grows in a lepidic pattern along alveoli and does not metastasize. A549 (moderate OPN) is a well-studied adenocarcinoma cell line, and H460 (high OPN) is a large cell undifferentiated aggressive cell type with high malignant potential and resistance to chemotherapy.

The inventors have demonstrated that over-expression of OPN's c-isoform (OPNc) did not confer increased malignant behavior in NSCLC cell lines, as seen with expression of the full-length isoform (Goparaju et al. “Functional Heterogeneity of Osteopontin Isoforms in Non-small Cell Lung Cancer,” J Thorac Oncol 5:1516-23 (2010) and Blasberg et al., “Lung Cancer Osteopontin Isoforms Exhibit Angiogenic Functional Heterogeneity,” The Journal of Thoracic and Cardiovascular Surgery 139:1587-93 (2010), which are hereby incorporated by reference in their entirety). The only structural difference between a- and c-isoforms of OPN is a 27 amino acid sequence (exon 4) (SEQ ID NO:3) in the amino end of the molecule. As noted above, strikingly, although exon 4 has been implicated in OPN-mediated lymphocyte activation in rheumatoid arthritis (Cao et al., “A Novel Functional Motif of Osteopontin for Human Lymphocyte Migration and Survival,” Mol Immunol 45:3683-92 (2008) and Dai et al., “A Functional Motif QLYxxYP is Essential for Osteopontin Induced T Lymphocyte Activation and Migration,” Biochem Biophys Res Commun 380:715-20 (2009), which are hereby incorporated by reference in their entirety), it has not been previously recognized as an important regulatory sequence for OPN in oncology. Likewise, the mechanisms for the functional differences between the isoforms have not been previously elucidated. Consequently, this experiment and those in the Examples that follow explore the molecular mechanisms conferred by cell binding of OPNa through exon 4 in NSCLC and targets this interaction for therapeutic benefit.

Further, Cisplatin resistance by NSCLC is a vital medical problem resulting from deregulation of cell death and survival pathways. The three naturally occurring OPN splice variants are differentially expressed in NSCLC and have divergent impact on malignant potential. OPNa, the full length protein, is disproportionately expressed in tumors and cell lines, increasing invasion and decreasing apoptosis compared to the shorter splice variants. OPNa overexpression is also uniquely associated with down-stream gene-expression pattern consistent with EMT, a mechanism for cisplatin resistance in NSCLC. Conversely, OPNc, the shortest splice variant is not expressed in NSCLC tumors or cell lines, but inhibits invasion and platinum resistance when expressed. The critical RGD sequence is expressed in each of the OPN isoforms. The only structural difference between OPNa and OPNc is the transcription of exon 4 in the amino terminus. The present experiments demonstrate that the OPNa isoform alone contributes to the malignant potential in NSCLC and that targeting the structural difference between OPNa and OPNc will decrease invasion and cisplatin resistance.

### Mutation of Exon 4 of Osteopontin Abrogates OPNa-Mediated Malignant Behavior and Increases Chemotherapeutic Sensitivity

A 4-amino acid deletion (PDAV) was made in exon 4 of OPN by site-directed mutagenesis. The sequence was validated and functional impact evaluated by transfecting cDNA plasmids specific to OPNa, OPNc, PDAV deleted OPN, or empty vector control into three NSCLC cell lines: H358 (low endogenous OPN) and A549 and H460 (high endogenous OPN). Pooled populations of cells were used in scratch closure, proliferation, invasion, and cisplatin treatment assays.

More particularly, Blast analysis of exon 4 gene sequence indicated that a deletion of the PDAV segment had potential to disrupt functional motifs. The deletion of the cDNA sequence corresponding to PDAV was completed using tools from Stratgene (Agilent, Santa Cara, Calif.) and verified by sequencing. cDNA plasmids consistent with OPNa, OPNc, PDAV-deleted OPN, and pCMV2 empty vector for control were transfected into three NSCLC cell lines as previously described (Goparaju et al. “Functional Heterogeneity of Osteopontin Isoforms in Non-small Cell Lung Cancer,” J Thorac Oncol 5:1516-23 (2010), which is hereby incorporated by reference in its entirety). Pooled populations of transfected cells were then used in invasion, proliferation, soft-agar colony formation and migration assays as previously described (Goparaju et al. “Functional Heterogeneity of Osteopontin Isoforms in Non-small Cell Lung Cancer,” J Thorac Oncol 5:1516-23 (2010), which is hereby incorporated by reference in its entirety).

This study revealed the importance of a particular 4-amino acid (PDAV) sequence. In particular, deletion of the PDAV sequence resulted in an OPN mutant which, when expressed in NSCLC cell lines, failed to stimulate proliferation (FIG. 4A), migration (FIG. 4B), anchorage independent growth (FIG. 4C), and invasion (FIG. 4D). As is shown in FIG. 4, the PDAV deleted OPN activity mirrored that of OPNc expression, significantly decreasing proliferation, invasion, and migration and increasing cisplatin sensitivity compared to controls in cell lines with high endogenous OPN. More particularly, deletion of the PDAV sequence resulted in an OPN mutant which, when expressed in NSCLC cell lines, decreased cell viability at cisplatin concentrations of 0.1-0.8 uM/ml (FIG. 4E).

The fact that OPN acts extracellularly coupled with the fortuitous observation that retention of the PDAV sequences, rather than its deletion, leads to malignant behavior is unique and ideal for, inter alia, antibody or targeted small molecule therapies.

### Example 2

### An Exon 4-Specific Peptide Antagonizes OPNa-Mediated Malignant Properties and Increases Chemotherapeutic Sensitivity

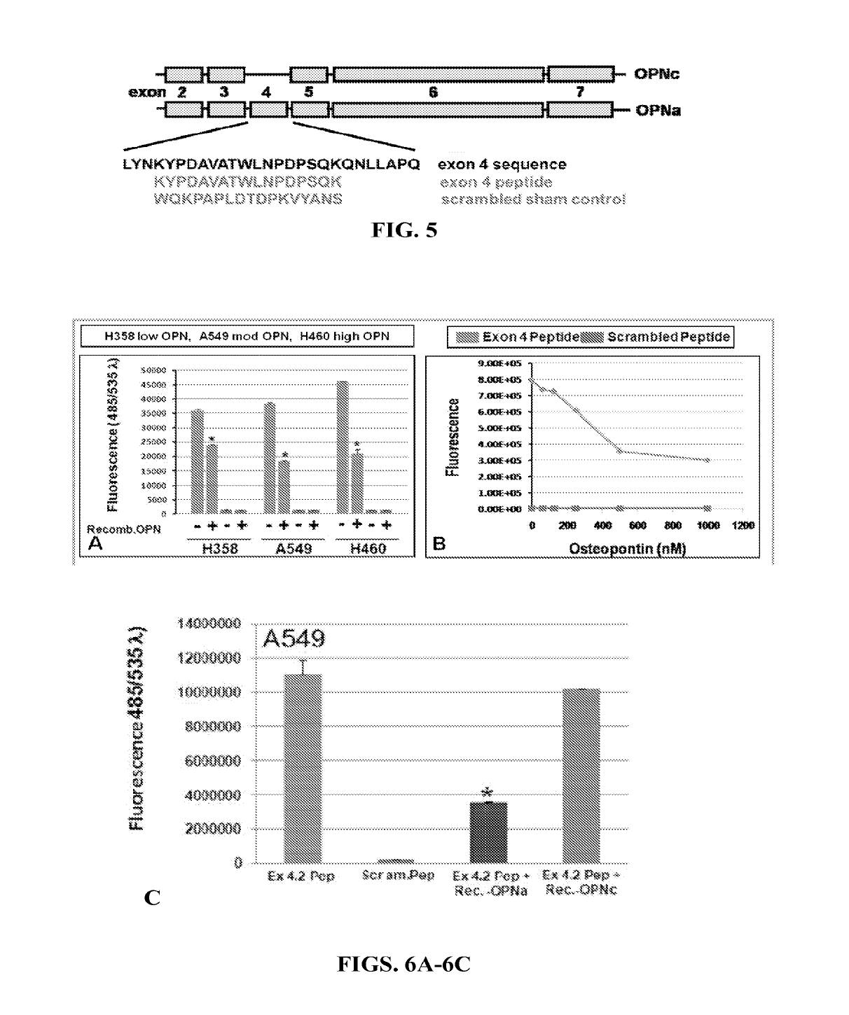

Based on the findings noted in the Examples above, a 17-amino acid peptide (SEQ ID NO:4) corresponding to the central region of exon 4 was created that included the PDAV (SEQ ID NO:5) sequence (FIG. 5). The peptide mimic (SEQ ID NO:4) and a scrambled sham (SEQ ID NO:23) were constructed and florescent labeled. Three NSCLC cell lines, H358 (low), A549 (moderate), and H460 (high endogenous OPN) were used in binding, proliferation, and invasion assays in presence of exon 4 mimic or sham. Cisplatin sensitivity was performed with peptides in A549.

More particularly, the peptide mimic sequence was selected that contained the PDAV region, along with a scrambled sham of the same length. Peptides were constructed and fluorescent (FAM) labeled by Think peptides (Proimmune, Sarasota, Fla.). NSCLC cell surface binding was assayed by seeding ten thousand NSCLC cells per well in a 96-well plate and allowing them to sit for overnight at 37° C. The cells were treated with 100 nM of FAM labeled Exon4 and FAM labeled scrambled peptide for 1 hour and then gently washed with 0.1% BSA in PBS for three times followed by treating with recombinant Osteopontin at 1 uM, 500 nM, 250 nM, 125 nM, 62.5 nM and 31.25 nM for 30 minutes. The cells are then washed thrice with 0.1% BSA in PBS and measured the fluorescence at 560/590λ.

In vitro invasion, migration, proliferation and soft agar colony formation assays were performed using the H358, A549 and H460 cell lines as previously described (Goparaju et al. “Functional Heterogeneity of Osteopontin Isoforms in Non-small Cell Lung Cancer,” J Thorac Oncol 5:1516-23 (2010), which is hereby incorporated by reference in its entirety) with 100 nM of exon 4 peptide or scrambled sham peptide in experimental medium. Experiments were performed in triplicates and comparisons of effect were made between peptide and sham within each cell line.

The effect of exon 4 peptide on sensitivity to cisplatin was evaluated by plating 10,000 A549 cells/well in a 96-well plate in triplicates. Five hours later, the medium was changed to DMEM containing 100 nM of exon 4 peptide, 100 nM of scrambled sham peptide, or no peptide. Fifteen minutes later, the medium was changed to DMEM containing 1.66, 0.83, 0.41, 0.20, 0.10, 0.05, and 0.025 mM of cisplatin for 48 h at 37° C. The cells were then assayed for survival by adding 20 ml of Cell Titer Blue reagent for 1 h. The fluorescence was read at 560/590λ.

Competitive binding assays employing the fluorescently labeled exon 4 peptide and recombinant full-length OPN (rOPNa, R&D, Minneapolis, Minn.) as competitor indicated that the exon 4 peptide specifically binds to the surface of the NSCLC cell lines, while the fluorescently labeled scrambled sham peptide does not (FIG. 6A). Further, increasing doses of recombinant OPN were able to compete the peptide off the cell surface (FIG. 6B). Specificity of exon-4 cell-surface interactions were validated by the fact that recombinant OPNc (rOPNc) does not disrupt fluorescent peptide binding to NSCLC cells (FIG. 6C). In particular, FIG. 6C shows results demonstrating that exon 4 peptide is displaced by molar equivalents of rOPNa, but the same concentrations of OPNc had no effect on cell surface binding.

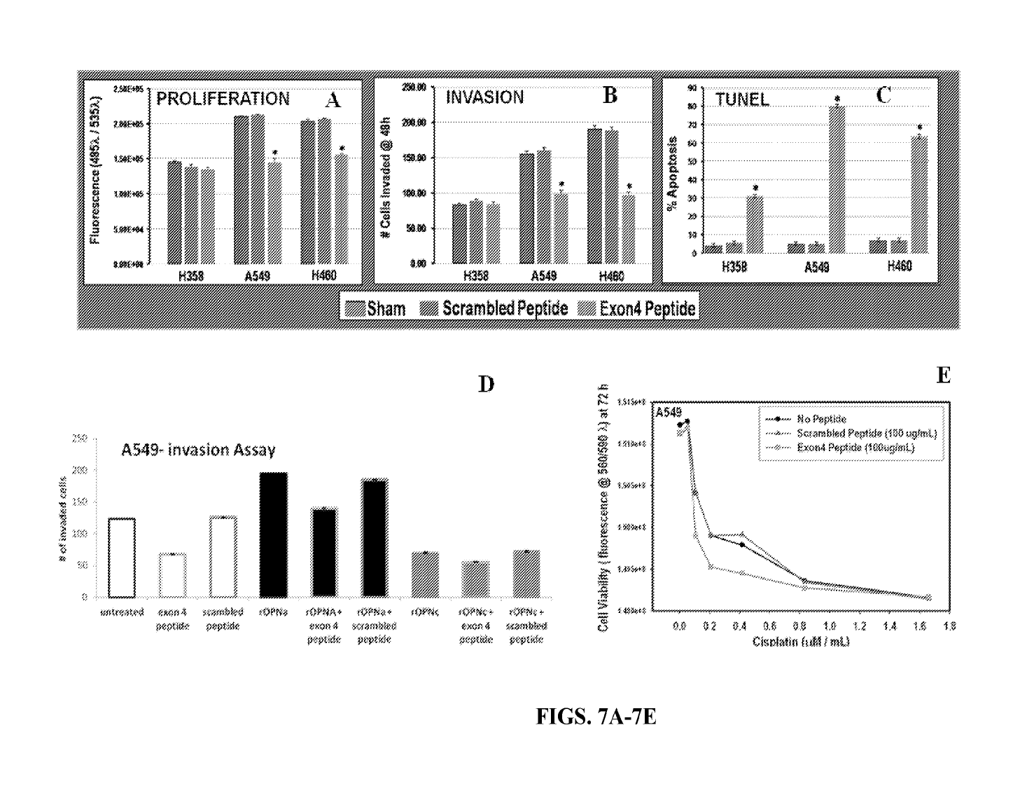

The addition of the exon 4 peptide to media decreased invasion (FIGS. 7B and 7D) and proliferation (FIG. 7A) and increased apoptosis (FIG. 7C) in cells with high endogenous OPN expression (A549 and H460). The results also indicate that exon 4 peptide has less impact on same functions in H358 cells with low endogenous OPN expression. FIG. 7D additionally demonstrates both rOPNc and the exon 4 peptide decrease invasive capacity, and the addition of the exon 4 peptide to exogenous rOPNa blocks its impact on invasion. FIG. 7E shows that exon 4 peptide increased cisplatin sensitivity, as indicated by the 2-fold decrease in cell viability compared to untreated or scrambled peptide at cisplatin concentrations of 0.1-0.8 μM/ml.

The effects of mutated OPN and the exon 4 peptide in H358, a NSCLC cell line with low endogenous OPN expression, were insightful. This cell line has low malignant potential; however, malignant properties are increased by over-expression of full-length OPNa, but not by over-expression of OPNc or the PDAV-deleted mutant. Further, the tumorigenic effects of full-length OPNa over-expression are abolished by addition of the exon 4 peptide in this cell line.

These competitive binding assays indicate that the exon 4 peptide associates with the same binding pocket as full-lengthen OPN. Yet functional assays indicate the peptide inhibits OPN actions. This indicates that the peptide contains the necessary sequence to bind, but not engage the binding pocket of a membrane-associated protein complex. In this way, the peptide antagonizes OPN exon 4 binding and activation through steric hindrance of the binding pocket. The exon 4 peptide described herein is a unique and powerful tool which is used to help understand cell surface interactions at OPN exon 4 and to elucidate mechanisms essential to the malignant properties conferred by OPN.

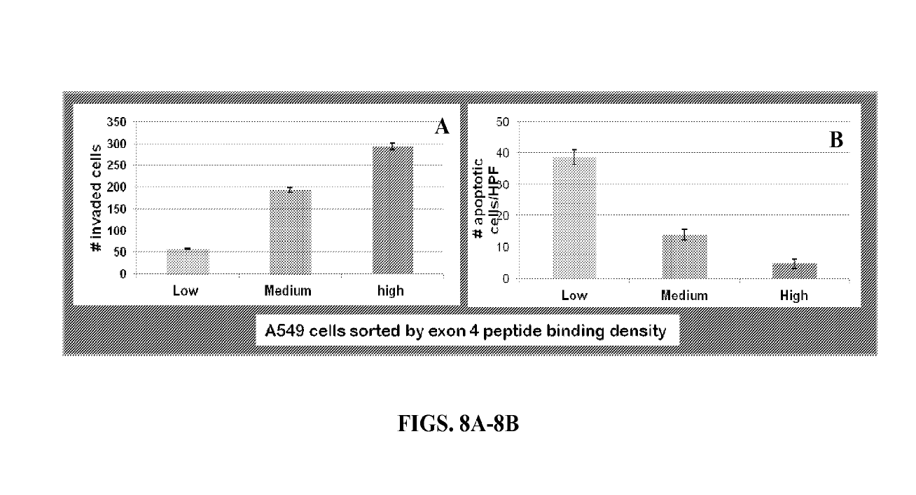

The exon 4 peptide was also used in a ligand sorting assay. A549 cells were sorted by fluorescence-activated cell sorting (“FACS”) into 3 groups (i.e., top 20%, mid 60%, and bottom 20%) based on the density of florescent labeled exon 4 peptide (see FIGS. 8A-8B). The sorted cells were washed and used in invasion and TUNEL assays. Sorted cells manifested increasing invasive properties and decreased rates of apoptosis as a function of the exon 4 binding complex.

### Example 3

### Mutation of Exon 4 of Osteopontin Abrogates OPNa-Mediated Epithelial-Mesenchymal Transition (“EMT”)

OPN is an important mediator of EMT (Saika et al. “Loss of Osteopontin Perturbs the Epithelial-mesenchymal Transition in an Injured Mouse Lens Epithelium,” Lab Invest 87:130-8 (2007); Pai et al., “S-Allylcysteine Inhibits Tumour Progression and the Epithelial-Mesenchymal Transition in a Mouse Xenograft Model of Oral Cancer,” Br J Nutr 1-11 (2011); and Bhattacharya et al., “Osteopontin Regulates Epithelial Mesenchymal Transition-Associated Growth of Hepatocellular Cancer in a Mouse Xenograft Model,” Ann Surg 255:319-25 (2012), which are hereby incorporated by reference in their entirety). OPN inhibition in vivo decrease tumor growth and the expression of EMT markers (Bhattacharya et al., “Osteopontin Regulates Epithelial Mesenchymal Transition-Associated Growth of Hepatocellular Cancer in a Mouse Xenograft Model,” Ann Surg 255:319-25 (2012), which is hereby incorporated by reference in its entirety). Affymetrix Gene Chip™ (Santa Clara, Calif.) expression profiles of A549 cells (high endogenous OPN) indicate that OPN-mediated EMT in NSCLC may depend on exon 4 binding. Over-expression of full length OPNa resulted in amplification of a large number of the molecular markers for EMT, including MMP-2, Snail-1, Snail-2, TGFβ receptor-1, MMP-9, N-cadherin, and vimentin, and a relative decrease in the expression of cytokeratin 18, and 20, desmoplakin and e-cadherin, consistent with activation of EMT pathways by OPNa (Lee et al., “The Epithelial-mesenchymal Transition: New Insights in Signaling, Development, and Disease,” The Journal of Cell Biology 172:973-81 (2006), which is hereby incorporated by reference in its entirety).

EMT is strongly associated with tumor progression (Soltermann et al., “Prognostic Significance of Epithelial-mesenchymal and Mesenchymal-epithelial Transition Protein Expression in Non-small Cell Lung Cancer,” Clin Cancer Res 14:7430-7 (2008), which is hereby incorporated by reference in its entirety) and cisplatin resistance in NSCLC (Zhuo et al., “Knockdown of Snail, a Novel Zinc Finger Transcription Factor, via RNA Interference Increases A549 Cell Sensitivity to Cisplatin via JNK/Mitochondrial Pathway,” Lung Cancer 62:8-14 (2008) and Zhuo et al., “Short Interfering RNA Directed Against TWIST, a Novel Zinc Finger Transcription Factor, Increases A549 Cell Sensitivity to Cisplatin via MAPK/Mitochondrial Pathway,” Biochem Biophys Res Commun 369:1098-102 (2008), which are hereby incorporated by reference in their entirety). The EMT expression pattern was validated by RT-QPCR in two additional NSCLC cell lines, H358 and H460 (Goparaju et al. “Functional Heterogeneity of Osteopontin Isoforms in Non-small Cell Lung Cancer,” J Thorac Oncol 5:1516-23 (2010), which is hereby incorporated by reference in its entirety). Gene arrays from cells expressing the OPNc or PDAV-deleted OPN do not have expression patterns consistent with EMT activation. The divergence in gene expression is consistent with in vitro functional data and implicates OPN activation of EMT as a regulatory step in NSCLC progression that is dependent on exon 4 interactions.

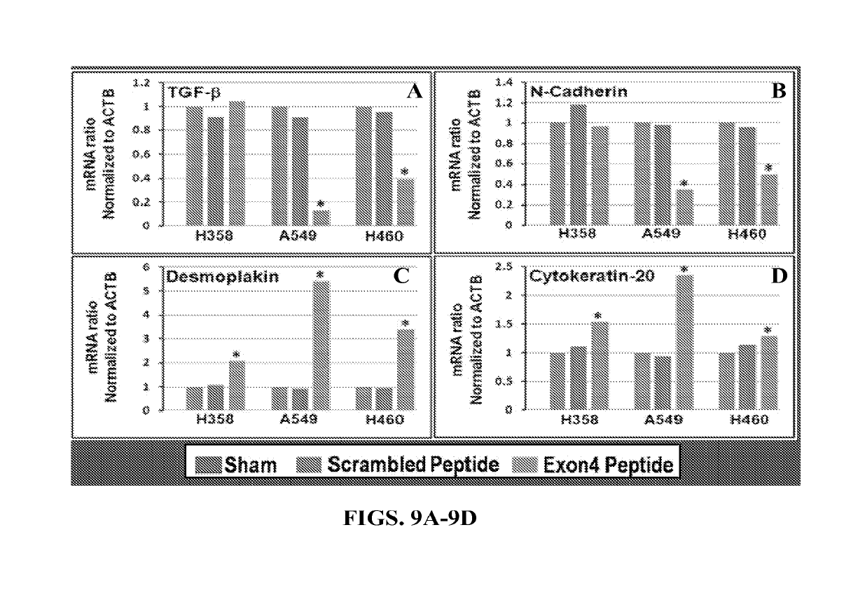

Here, mRNA was extracted from peptide treated cells and the relative expression of EMT targets evaluated by quantitative RT-PCR. As shown in FIGS. 9A to 9D, treatment with exon 4 peptide resulted in a significant decrease in the relative expression of TFGb1-r (FIG. 9A) and N-cadherin (FIG. 9B) and a significant increase in desmoplakin (FIG. 9C) and cytokeratin-20 (FIG. 9D) in the A549 and H460 cell lines, consistent with reversal of EMP pathways. The exon 4 peptide had a lesser impact on the same markers in the H358 cell line with low endogenous OPN.

Exon 4 of OPN has not previously been identified as a regulatory region essential for OPN's effect on malignant behavior. The present experiments provide evidence for an un-described cell surface receptor to this region. Further, the similarity in outcomes of functional assay between the OPN PDAV-deleted mutant and those performed in the presence of the exon 4 peptide, indicates that the peptide antagonizes a unique cell surface site which is essential for the increased malignant properties and avoidance of cisplatin induced apoptosis conferred by OPNa exon 4 in NSCLC. A small mimicking peptide to OPN exon 4 significantly decreased in vitro malignant behavior, suggesting interference with important regulatory pathways. This novel binding site serves as an ideal target for, inter alia, antibody or small molecule therapies in NSCLC.

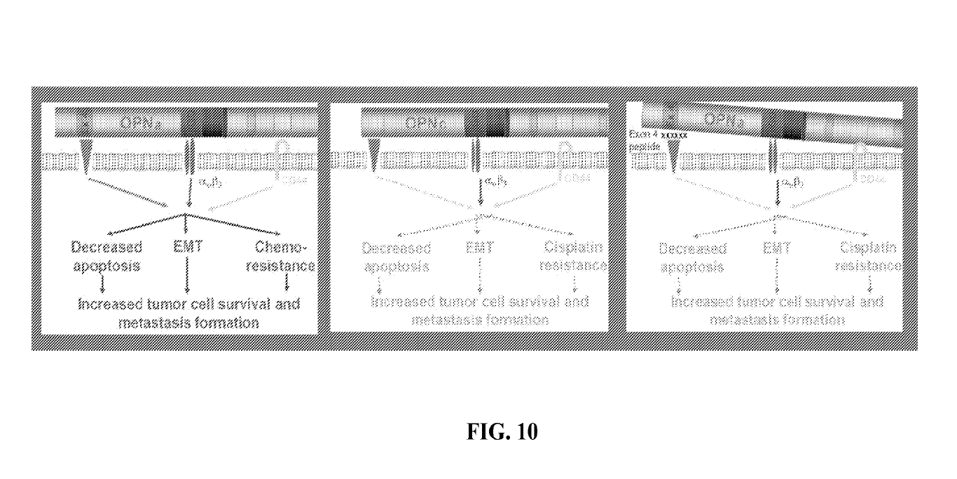

Without being bound by theory, these results indicate that the peptide mimic contains the necessary sequence to bind, but not engage the binding pocket of a membrane-associated protein. In this way, steric hindrance by the peptide can antagonize OPN exon 4 binding and activation of this same binding pocket (FIG. 10).

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A therapeutic comprising:
an osteopontin isoform a (“OPNa”) inhibitor, wherein the OPNa inhibitor blocks activity of extracellular OPNa exon 4 and is selected from the group consisting of:
(i) an exon-4 specific antibody or binding portion thereof;
(ii) a peptide mimic of OPNa exon 4 or a fragment thereof;
(iii) a nucleic acid aptamer that specifically binds to OPNa exon 4 or a fragment thereof; and
(iv) a peptide inhibitor that binds to OPNa exon 4 or a fragment thereof.

2. The therapeutic according to claim 1, wherein the OPNa inhibitor blocks OPNa exon 4 cell surface receptor binding activity.

3. The therapeutic according to claim 1, wherein OPNa exon 4 comprises the amino acid sequence of LYNKYPDAVATWLNPDPSQKQNLLAPQ (SEQ ID NO:3).

4. The therapeutic according to claim 1, wherein the OPNa inhibitor is an exon-4 specific antibody or binding fragment thereof that recognizes at least a portion of OPNa exon 4 comprising the amino acid sequence of PDAV (SEQ ID NO:5).

5. The therapeutic according to claim 1, wherein the OPNa inhibitor is an exon-4 specific antibody or binding fragment thereof that recognizes at least a portion of OPNa exon 4 comprising the amino acid sequence of KYPDAVATWLNPDPSQK (SEQ ID NO:4).

6. The therapeutic according to claim 1, wherein the OPNa inhibitor is an exon-4 specific antibody or binding fragment thereof that recognizes exon 4 comprising the amino acid sequence LYNKYPDAVATWLNPDPSQKQNLLAPQ (SEQ ID NO:3).

7. The therapeutic according to claim 1, wherein the OPNa inhibitor comprises a peptide mimic of OPNa exon 4 or a fragment thereof.

8. The therapeutic according to claim 7, wherein the peptide mimic of OPNa exon 4 has the formula (R₁)ₓ-R₂-(R₃)_{y}, wherein
R₁ is an amino acid sequence comprising from 1 to 30 amino acids, wherein each amino acid is independently selected from the group consisting of naturally occurring amino acids and amino acid analogs;
R₂ is PDAV (SEQ ID NO:5);
R₃ comprises is an amino acid sequence comprising from 1 to 30 amino acids, wherein each amino acid is independently selected from the group consisting of naturally occurring amino acids and amino acid analogs; and
x and y are independently selected and equal to zero or one.

9. The therapeutic according to claim 8, wherein R₁ is selected from the group consisting of Y, KY, NKY, YNKY (SEQ ID NO:6), and LYNKY (SEQ ID NO:7).

10. The therapeutic according to claim 8, wherein R₃ is selected from the group consisting of A, AT, ATW, ATWL (SEQ ID NO:8), ATWLN (SEQ ID NO:9), ATWLNP (SEQ ID NO:10), ATWLNPD (SEQ ID NO:11), ATWLNPDP (SEQ ID NO:12), ATWLNPDPS (SEQ ID NO:13), ATWLNPDPSQ (SEQ ID NO:14), ATWLNPDPSQK (SEQ ID NO:15), ATWLNPDPSQKQ (SEQ ID NO:16), ATWLNPDPSQKQN (SEQ ID NO:17), ATWLNPDPSQKQNL (SEQ ID NO:18), ATWLNPDPSQKQNLL (SEQ ID NO:19), ATWLNPDPSQKQNLLA (SEQ ID NO:20), ATWLNPDPSQKQNLLAP (SEQ ID NO:21), and ATWLNPDPSQKQNLLAPQ (SEQ ID NO:22).

11. The therapeutic according to claim 8, wherein the peptide mimic of OPNa exon 4 comprises the amino acid sequence selected from the group consisting of PDAV (SEQ ID NO:5); KYPDAVATWLNPDPSQK (SEQ ID NO:4); and LYNKYPDAVATWLNPDPSQKQNLLAPQ (SEQ ID NO:3).

12. The therapeutic according to claim 1, wherein the OPNa inhibitor comprises a nucleic acid aptamer that specifically binds to OPNa exon 4 or a fragment thereof.

13. The therapeutic according to claim 12, wherein the nucleic acid aptamer that specifically binds to at least a fragment of OPNa exon 4 comprising the amino acid sequence PDAV (SEQ ID NO:5).

14. The therapeutic according to claim 1, wherein the OPNa inhibitor comprises a peptide inhibitor that binds to OPNa exon 4 or a fragment thereof.

15. The therapeutic according to claim 14, wherein the peptide inhibitor specifically binds to at least a fragment of OPNa exon 4 comprising the amino acid sequence PDAV (SEQ ID NO:5).

16. The therapeutic according to claim 1 further comprising:
a cancer therapeutic.

17. The therapeutic according to claim 16, wherein the cancer therapeutic is a chemotherapeutic selected from the group consisting of alkylating agents, antimetabolites, anthracyclines, antitumor antibiotics, platinium-based chemotherapeutics, and plant alkaloids.

18. The therapeutic according to claim 17, wherein the chemotherapeutic is a platinum-based chemotherapeutic.

19. The therapeutic according to claim 16, wherein the chemotherapeutic is selected from the group consisting of oxaliplatin, cyclophosphamide, ifosfamide, thiotepa, melphalan, busulfan, nimustine, ranimustine, dacarbazine, procarbazine, temozolomide, cisplatin, carboplatin, nedaplatin, methotrexate, pemetrexed, fluorouracil, tegaful/uracil, doxifluridine, tegaful/gimeracil/oteracil, capecitabine, cytarabine, enocitabine, gemcitabine, 6-mercaptopurine, fuludarabin, pentostatin, cladribine, hydroxyurea, doxorubicin, epirubicin, daunorubicin, idarubicine, pirarubicin, mitoxantrone, amurubicin, actinomycin D, bleomycine, pepleomycin, mytomycin C, aclarubicin, zinostatin, vincristine, vindesine, vinblastine, vinorelbine, paclitaxel, docetaxel, irinotecan, irinotecan active metabolite (SN-38), nogitecan (topotecan), etoposide, prednisolone, dexamethasone, tamoxifen, toremifene, medroxyprogesterone, anastrozole, exemestane, letrozole, rituximab, imatinib, gefitinib, gemtuzumab ozogamicin, bortezomib, erlotinib, cetuximab, bevacizumab, sunitinib, sorafenib, dasatinib, panitumumab, asparaginase, tretinoin, arsenic trioxide, salts thereof, active metabolites thereof, and combinations thereof.

20. The therapeutic according to claim 1 further comprising:
a pharmaceutically acceptable carrier.

21. A method of inhibiting tumor growth and/or metastasis in a subject comprising:
selecting a subject having a tumor, wherein the tumor cells express OPNa exon 4 and
administering to the selected subject an OPNa inhibitor.

22. The method according to claim 21, wherein the OPNa inhibitor blocks activity of extracellular OPNa exon 4 and is selected from the group consisting of:
(i) an exon-4 specific antibody or binding portion thereof;
(ii) a peptide mimic of OPNa exon 4 or a fragment thereof;
(iii) a nucleic acid aptamer that specifically binds to OPNa exon 4 or a fragment thereof; and
(iv) a peptide inhibitor that binds to OPNa exon 4 or a fragment thereof.

23. A method of inhibiting tumor growth and/or metastasis in a subject comprising:
selecting a subject having a tumor, wherein the tumor cells express OPNa exon 4 and
administering to the selected subject an OPNa inhibitor and a cancer therapeutic.

24. The method according to claim 23, wherein the OPNa inhibitor blocks activity of extracellular OPNa exon 4 and is selected from the group consisting of:
(i) an exon-4 specific antibody or binding portion thereof;
(ii) a peptide mimic of OPNa exon 4 or a fragment thereof;
(iii) a nucleic acid aptamer that specifically binds to OPNa exon 4 or a fragment thereof; and
(iv) a peptide inhibitor that binds to OPNa exon 4 or a fragment thereof.

25. A method of treating a subject with chemotherapeutic resistance comprising:
selecting a subject having chemotherapeutic resistance and
administering to the selected subject an OPNa inhibitor and a cancer therapeutic.

26. The method according to claim 25, wherein the OPNa inhibitor blocks activity of extracellular OPNa exon 4 and is selected from the group consisting of:
(i) an exon-4 specific antibody or binding portion thereof;
(ii) a peptide mimic of OPNa exon 4 or a fragment thereof;
(iii) a nucleic acid aptamer that specifically binds to OPNa exon 4 or a fragment thereof; and
(iv) a peptide inhibitor that binds to OPNa exon 4 or a fragment thereof.

27. A method for increasing tumor cell sensitivity to a cancer therapeutic in a subject comprising:
selecting a subject in need of increased tumor cell sensitivity to a cancer therapeutic;
providing an OPNa inhibitor;
providing the cancer therapeutic; and
administering to said subject the OPNa inhibitor and the cancer therapeutic under conditions effective to increase tumor cell sensitivity to the cancer therapeutic in the selected subject as compared to administration of the cancer therapeutic alone.

28. The method according to claim 21, wherein the OPNa inhibitor blocks activity of extracellular OPNa exon 4.

29. The method according to claim 28, wherein the OPNa inhibitor blocks OPNa exon 4 cell surface receptor binding activity.

30. The method according to claim 28, wherein OPNa exon 4 comprises the amino acid sequence of LYNKYPDAVATWLNPDPSQKQNLLAPQ (SEQ ID NO:3).

31. The method according to claim 21, wherein the OPNa inhibitor is an exon-4 specific antibody or binding fragment thereof that recognizes at least a portion of OPNa exon 4 comprising the amino acid sequence of PDAV (SEQ ID NO:5).

32. The method according to claim 21, wherein the OPNa inhibitor is an exon-4 specific antibody or binding fragment thereof that recognizes at least a portion of OPNa exon 4 comprising the amino acid sequence of KYPDAVATWLNPDPSQK (SEQ ID NO:4).

33. The method according to claim 21, wherein the OPNa inhibitor is an exon-4 specific antibody or binding fragment thereof that recognizes OPNa exon 4 comprising the amino acid sequence of LYNKYPDAVATWLNPDPSQKQNLLAPQ (SEQ ID NO:3).

34. The method according to claim 21, wherein the OPNa inhibitor comprises a peptide mimic of OPNa exon 4 or a fragment thereof.

35. The method according to claim 34, wherein the peptide mimic of OPNa exon 4 has the formula (R₁)ₓ-R₂-(R₃)_{y}, wherein
R₁ is an amino acid sequence comprising from 1 to 30 amino acids, wherein each amino acid is independently selected from the group consisting of naturally occurring amino acids and amino acid analogs;
R₂ is PDAV (SEQ ID NO:5);
R₃ comprises is an amino acid sequence comprising from 1 to 30 amino acids, wherein each amino acid is independently selected from the group consisting of naturally occurring amino acids and amino acid analogs; and
x and y are independently selected and equal to zero or one.

36. The method according to claim 35, wherein R₁ is selected from the group consisting of Y, KY, NKY, YNKY (SEQ ID NO:6), and LYNKY (SEQ ID NO:7).

37. The method according to claim 35, wherein R₃ is selected from the group consisting of A, AT, ATW, ATWL (SEQ ID NO:8), ATWLN (SEQ ID NO:9), ATWLNP (SEQ ID NO:10), ATWLNPD (SEQ ID NO:11), ATWLNPDP (SEQ ID NO:12), ATWLNPDPS (SEQ ID NO:13), ATWLNPDPSQ (SEQ ID NO:14), ATWLNPDPSQK (SEQ ID NO:15), ATWLNPDPSQKQ (SEQ ID NO:16), ATWLNPDPSQKQN (SEQ ID NO:17), ATWLNPDPSQKQNL (SEQ ID NO:18), ATWLNPDPSQKQNLL (SEQ ID NO:19), ATWLNPDPSQKQNLLA (SEQ ID NO:20), ATWLNPDPSQKQNLLAP (SEQ ID NO:21), and ATWLNPDPSQKQNLLAPQ (SEQ ID NO:22).

38. The method according to claim 35, wherein the peptide mimic of OPNa exon 4 comprises the amino acid sequence selected from the group consisting of PDAV (SEQ ID NO:5); KYPDAVATWLNPDPSQK (SEQ ID NO:4); and LYNKYPDAVATWLNPDPSQKQNLLAPQ (SEQ ID NO:3).

39. The method according to claim 21, wherein the OPNa inhibitor comprises a nucleic acid aptamer that specifically binds to OPNa exon 4 or a fragment thereof.

40. The method according to claim 39, wherein the nucleic acid aptamer that specifically binds to at least a fragment of OPNa exon 4 comprising the amino acid sequence PDAV (SEQ ID NO:5).

41. The method according to claim 21, wherein the OPNa inhibitor comprises a peptide inhibitor that binds to OPNa exon 4 or a fragment thereof.

42. The method according to claim 21, wherein the peptide inhibitor specifically binds to at least a fragment of OPNa exon 4 comprising the amino acid sequence PDAV (SEQ ID NO:5).

43. The method according to claim 23, wherein the cancer therapeutic is a chemotherapeutic selected from the group consisting of alkylating agents, antimetabolites, anthracyclines, antitumor antibiotics, platinium-based chemotherapeutics, and plant alkaloids.

44. The method according to claim 43, wherein the chemotherapeutic is a platinum-based chemotherapeutic.

45. The method according to claim 44, wherein the chemotherapeutic is cisplatin.

46. The method according to claim 23, wherein the cancer therapeutic is selected from the group consisting of oxaliplatin, cyclophosphamide, ifosfamide, thiotepa, melphalan, busulfan, nimustine, ranimustine, dacarbazine, procarbazine, temozolomide, cisplatin, carboplatin, nedaplatin, methotrexate, pemetrexed, fluorouracil, tegaful/uracil, doxifluridine, tegaful/gimeracil/oteracil, capecitabine, cytarabine, enocitabine, gemcitabine, 6-mercaptopurine, fuludarabin, pentostatin, cladribine, hydroxyurea, doxorubicin, epirubicin, daunorubicin, idarubicine, pirarubicin, mitoxantrone, amurubicin, actinomycin D, bleomycine, pepleomycin, mytomycin C, aclarubicin, zinostatin, vincristine, vindesine, vinblastine, vinorelbine, paclitaxel, docetaxel, irinotecan, irinotecan active metabolite (SN-38), nogitecan (topotecan), etoposide, prednisolone, dexamethasone, tamoxifen, toremifene, medroxyprogesterone, anastrozole, exemestane, letrozole, rituximab, imatinib, gefitinib, gemtuzumab ozogamicin, bortezomib, erlotinib, cetuximab, bevacizumab, sunitinib, sorafenib, dasatinib, panitumumab, asparaginase, tretinoin, arsenic trioxide, salts thereof, active metabolites thereof, and combinations thereof.

47. The method according to claim 23, wherein the OPNa inhibitor and the cancer therapeutic are administered simultaneously.

48. The method according to claim 23, wherein the OPNa inhibitor is administered to the subject before the cancer therapeutic is administered.

49. The method according to claim 23, wherein the OPNa inhibitor is administered to the subject after the cancer therapeutic is administered.

50. The method according to claim 21, wherein said administering is intravenous or subcutaneous.

51. The method according to claim 21, wherein the selected subject is mammal.

52. The method according to claim 51, wherein the selected subject is human.

53. The method according to claim 21, wherein the selected subject has non-small cell lung cancer.


SEQUENCE LISTING (23 sequences)

SEQ ID NO: 1 - DNA, 1641 bp, Homo sapiens
ctccctgtgttggtggaggatgtctgcagcagcatttaaattctgggagggcttggttgtcagcagcagcaggaggaggcagagcacagcatcgtcgggaccagactcgtctcaggccagttgcagccttctcagccaaacgccgaccaaggaaaactcactaccatgagaattgcagtgatttgcttttgcctcctaggcatcacctgtgccataccagttaaacaggctgattctggaagttctgaggaaaagcagctttacaacaaatacccagatgctgtggccacatggctaaaccctgacccatctcagaagcagaatctcctagccccacagaatgctgtgtcctctgaagaaaccaatgactttaaacaagagacccttccaagtaagtccaacgaaagccatgaccacatggatgatatggatgatgaagatgatgatgaccatgtggacagccaggactccattgactcgaacgactctgatgatgtagatgacactgatgattctcaccagtctgatgagtctcaccattctgatgaatctgatgaactggtcactgattttcccacggacctgccagcaaccgaagttttcactccagttgtccccacagtagacacatatgatggccgaggtgatagtgtggtttatggactgaggtcaaaatctaagaagtttcgcagacctgacatccagtaccctgatgctacagacgaggacatcacctcacacatggaaagcgaggagttgaatggtgcatacaaggccatccccgttgcccaggacctgaacgcgccttctgattgggacagccgtgggaaggacagttatgaaacgagtcagctggatgaccagagtgctgaaacccacagccacaagcagtccagattatataagcggaaagccaatgatgagagcaatgagcattccgatgtgattgatagtcaggaactttccaaagtcagccgtgaattccacagccatgaatttcacagccatgaagatatgctggttgtagaccccaaaagtaaggaagaagataaacacctgaaatttcgtatttctcatgaattagatagtgcatcttctgaggtcaattaaaaggagaaaaaatacaatttctcactttgcatttagtcaaaagaaaaaatgctttatagcaaaatgaaagagaacatgaaatgcttctttctcagtttattggttgaatgtgtatctatttgagtctggaaataactaatgtgtttgataattagtttagtttgtggcttcatggaaactccctgtaaactaaaagcttcagggttatgtctatgttcattctatagaagaaatgcaaactatcactgtattttaatatttgttattctctcatgaatagaaatttatgtagaagcaaacaaaatacttttacccacttaaaaagagaatataacattttatgtcactataatcttttgttttttaagttagtgtatattttgttgtgattatctttttgtggtgtgaataaatcttttatcttgaatgtaataagaatttggtggtgtcaattgcttatttgttttcccacggttgtccagcaattaataaaacataaccttttttactgcctaaaaaaaaaaaaaaaaa

SEQ ID NO: 2 - protein, 314 aa, Homo sapiens
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 3 - protein, 27 aa, Artificial
  Peptide mimic of OPNa exon 4
LYNKYPDAVATWLNPDPSQKQNLLAPQ

SEQ ID NO: 4 - protein, 17 aa, Artificial
  Peptide mimic of OPNa exon 4
KYPDAVATWLNPDPSQK

SEQ ID NO: 5 - protein, 4 aa, Artificial
  R2 of peptide mimic of OPNa exon 4
PDAV

SEQ ID NO: 6 - protein, 4 aa, Artificial
  R1 of peptide mimic of OPNa exon 4
YNKY

SEQ ID NO: 7 - protein, 5 aa, Artificial
  R1 of peptide mimic of OPNa exon 4
LYNKY

SEQ ID NO: 8 - protein, 4 aa, Artificial
  R3 of peptide mimic of OPNa exon 4
ATWL

SEQ ID NO: 9 - protein, 5 aa, Artificial
  R3 of peptide mimic of OPNa exon 4
ATWLN

SEQ ID NO: 10 - protein, 6 aa, Artificial
  R3 of peptide mimic of OPNa exon 4
ATWLNP

SEQ ID NO: 11 - protein, 7 aa, Artificial
  R3 of peptide mimic of OPNa exon 4
ATWLNPD

SEQ ID NO: 12 - protein, 8 aa, Artificial
  R3 of peptide mimic of OPNa exon 4
ATWLNPDP

SEQ ID NO: 13 - protein, 9 aa, Artificial
  R3 of peptide mimic of OPNa exon 4
ATWLNPDPS

SEQ ID NO: 14 - protein, 10 aa, Artificial
  R3 of peptide mimic of OPNa exon 4
ATWLNPDPSQ

SEQ ID NO: 15 - protein, 11 aa, Artificial
  R3 of peptide mimic of OPNa exon 4
ATWLNPDPSQK

SEQ ID NO: 16 - protein, 12 aa, Artificial
  R3 of peptide mimic of OPNa exon 4
ATWLNPDPSQKQ

SEQ ID NO: 17 - protein, 13 aa, Artificial
  R3 of peptide mimic of OPNa exon 4
ATWLNPDPSQKQN

SEQ ID NO: 18 - protein, 14 aa, Artificial
  R3 of peptide mimic of OPNa exon 4
ATWLNPDPSQKQNL

SEQ ID NO: 19 - protein, 15 aa, Artificial
  R3 of peptide mimic of OPNa exon 4
ATWLNPDPSQKQNLL

SEQ ID NO: 20 - protein, 16 aa, Artificial
  R3 of peptide mimic of OPNa exon 4
ATWLNPDPSQKQNLLA

SEQ ID NO: 21 - protein, 17 aa, Artificial
  R3 of peptide mimic of OPNa exon 4
ATWLNPDPSQKQNLLAP

SEQ ID NO: 22 - protein, 18 aa, Artificial
  R3 of peptide mimic of OPNa exon 4
ATWLNPDPSQKQNLLAPQ

SEQ ID NO: 23 - protein, 17 aa, Artificial
  Scrambled Sham Peptide
WQKPAPLDTDPKVYANS